SEQUENCE LISTING (42 sequences)

SEQ ID NO: 1 - DNA, 11 bp, Homo sapiens
tgccacaaagc

SEQ ID NO: 2 - DNA, 11 bp, Homo sapiens
tgccataaagc

SEQ ID NO: 3 - DNA, 11 bp, Homo sapiens
gactgcagtgg

SEQ ID NO: 4 - DNA, 11 bp, Homo sapiens
gactggagtgg

SEQ ID NO: 5 - DNA, 11 bp, Homo sapiens
atgattcagag

SEQ ID NO: 6 - DNA, 11 bp, Homo sapiens
atgatgcagag

SEQ ID NO: 7 - DNA, 20 bp, Homo sapiens
ggctcacctggacaacctca

SEQ ID NO: 8 - DNA, 22 bp, Homo sapiens
aaagtgatgggccagcacacag

SEQ ID NO: 9 - DNA, 20 bp, Homo sapiens
aaagcacctggatgatctca

SEQ ID NO: 10 - DNA, 21 bp, Homo sapiens
aaaacggtcaccagcacattt

SEQ ID NO: 11 - DNA, 19 bp, Homo sapiens
cacatggcaagaaggtgct

SEQ ID NO: 12 - DNA, 24 bp, Homo sapiens
gcagaataaagcctatccttgaaa

SEQ ID NO: 13 - DNA, 21 bp, Homo sapiens
caagcccgcctttgctaagct

SEQ ID NO: 14 - DNA, 24 bp, Homo sapiens
ctccttgccaaagtgagtagccag

SEQ ID NO: 15 - DNA, 19 bp, Homo sapiens
gagggagcctgagaaacgg

SEQ ID NO: 16 - DNA, 20 bp, Homo sapiens
gtcgggagtgggtaatttgc

SEQ ID NO: 17 - DNA, 20 bp, Homo sapiens
tcccaagtctggaaagcgtc

SEQ ID NO: 18 - DNA, 20 bp, Homo sapiens
gcacatctgttcgattcggg

SEQ ID NO: 19 - DNA, 20 bp, Homo sapiens
tgccccaaacaggaacacat

SEQ ID NO: 20 - DNA, 20 bp, Homo sapiens
attctgcactcatcccaggc

SEQ ID NO: 21 - DNA, 23 bp, Homo sapiens
aagtcattggaacctggagtttg

SEQ ID NO: 22 - DNA, 20 bp, Homo sapiens
gttctctgctgcttgcgttt

SEQ ID NO: 23 - DNA, 20 bp, Homo sapiens
tcactggtacgcagggtttt

SEQ ID NO: 24 - DNA, 21 bp, Homo sapiens
tattgagttgtggggactggc

SEQ ID NO: 25 - DNA, 26 bp, Homo sapiens
attcccgtttttatgaaatcaacttt

SEQ ID NO: 26 - DNA, 30 bp, Homo sapiens
tcttagaaagcctttacaatttcctttatc

SEQ ID NO: 27 - DNA, 23 bp, Homo sapiens
agcttagtgatacttgtgggcca

SEQ ID NO: 28 - DNA, 22 bp, Homo sapiens
gctcggcacatgtcccatccag

SEQ ID NO: 29 - DNA, 28 bp, Homo sapiens
gtcagtgagtctaggcaagatgttggcc

SEQ ID NO: 30 - DNA, 28 bp, Homo sapiens
aaaaaatgtggaattagacccaggaatg

SEQ ID NO: 31 - DNA, 20 bp, Homo sapiens
gggtgtgtatttgtctgcca

SEQ ID NO: 32 - DNA, 22 bp, Homo sapiens
ttgccatacctcatatccttag

SEQ ID NO: 33 - DNA, 18 bp, Homo sapiens
cttaggcatccacaaggg

SEQ ID NO: 34 - DNA, 25 bp, Homo sapiens
agcaaggatggttcttaaggaaggg

SEQ ID NO: 35 - DNA, 21 bp, Homo sapiens
atccatgatctctaaccttgc

SEQ ID NO: 36 - DNA, 30 bp, Homo sapiens
aatttgaagatacagcttgcctccgataag

SEQ ID NO: 37 - DNA, 22 bp, Homo sapiens
gggttcatctttattgtctcct

SEQ ID NO: 38 - DNA, 28 bp, Homo sapiens
ccaccccgataaagatttttctccatca

SEQ ID NO: 39 - DNA, 26 bp, Homo sapiens
attaaccaatggtatctttctgagca

SEQ ID NO: 40 - DNA, 26 bp, Homo sapiens
ccaaatgggtgactgtagggttgaga

SEQ ID NO: 41 - DNA, 23 bp, Homo sapiens
cgtgagagcatacttcctggttc

SEQ ID NO: 42 - DNA, 21 bp, Homo sapiens
acaccagagaggtcttgccct

(19) United States
(12) Patent Application Publication
(10) Pub. No.: US 2022/0347186 A1
(43) Pub. Date: Nov. 3, 2022

(54) METHODS AND COMPOSITIONS FOR TREATING SICKLE CELL DISEASE AND THALASSEMIA

(71) Applicant: THE UNIVERSITY OF CHICAGO, Chicago (US)

(72) Inventors: Lucy A. Godley, Chicago, IL (US); John Z. Cao, Chicago, IL (US); Amittha Wickrema, Chicago, IL (US)

(21) Appl. No.: 17/763,445

(22) PCT Filed: Sep. 25, 2020
(86) PCT No.: PCT/US2020/052842

§ 371 Date: Mar. 24, 2022

Related U.S. Application Data
(60) Provisional application No. 62/907,087, filed on Sep. 27, 2019.

(51) Int. Cl. A61K 31/55; A61P 7/06; A61K 31/444; A61K 31/17; A61K 31/4439

(52) U.S. Cl. CPC A61K 31/55; A61P 7/06; A61K 31/444; A61K 31/17; A61K 31/4439

(57) ABSTRACT

This disclosure relates to methods and compositions for treating sickle cell anemia or thalassemia in a subject in need thereof. As described herein in a first aspect the present disclosure provides a method of treating an inherited blood disorder in a subject In need thereof comprising administering to the subject a therapeutically effective amount of a bromodomain and extra-terminal motif (BET) protein inhibitor.

### BACKGROUND OF DISCLOSURE

### Field of Invention

This disclosure relates to methods for treatment of sickle cell anemia and thalassemia.

### Technical Background

Genetic (inherited) blood disorders are blood diseases that are passed from chromosomes of parents to those of their children. Some such diseases are severe and can cause life-long health challenges. Disorders affecting hemoglobin, a protein in red blood cells that carries oxygen throughout the body, can be particularly challenging to address. A deficiency or mutation in hemoglobin often leads to anemia, a condition in which a patient can feel tired, weak, or short of breath. Severe anemia can cause organ damage and death.

Sickle cell disease (SCD) is a group of inherited red blood cell disorders. In patients with SCD, the hemoglobin is mutated and forms into stiff rods within the red blood cells. This changes the shape of the red blood cells that carry the protein: healthy cells are disc-shaped, but SCD-affected hemoglobin causes cells to be a crescent (sickle) shape.

The sickle-shaped cells are not flexible, and many of them burst apart as they move through blood vessels. Sickle cells usually only last 10 to 20 days, instead of the normal 90-120 day lifespan of red blood cells. The patient's body may have trouble making enough new cells to replace the ones that are lost prematurely. The resulting deficiency of red blood cells causes anemia. The sickle-shaped cells can also stick to vessel walls, causing a blockage that slows or stops the flow of blood. When this happens, oxygen cannot reach nearby tissues. The lack of oxygen can cause attacks of sudden, severe pain called pain crises and/or strokes when the brain is deprived of oxygen. These attacks can occur without warning, and patients are often hospitalized for these pain crisis events. Bone marrow transplants offer the only opportunity for cure and are usually reserved for young patients.

Thalassemia is another inherited blood disorder in which the body is unbalanced in its production of the hemoglobin chains. Because of this imbalance, the hemoglobin protein does not form normally, causing red blood cells to not function properly and last for shorter periods of time, leading to anemia. The disorder can cause the bone marrow to expand, leading to bone deformities. Some thalassemia patients require regular blood transfusions.

In light of the continued burden of inherited blood disorders on families, there is a need for new treatments to address inherited blood disorders, such as sickle cell anemia and thalassemia.

### SUMMARY OF THE DISCLOSURE

This disclosure describes methods and compositions for treating inherited blood disorders, including sickle cell anemia and thalassemia.

As described below, in a first aspect the present disclosure provides a method of treating an inherited blood disorder in a subject in need thereof, comprising administering to the subject a therapeutically effective amount of a bromodomain and extra-terminal motif (BET) protein inhibitor.

In one embodiment of the first aspect, the inherited blood disorder is thalassemia. In one embodiment, the thalassemia is alpha thalassemia. In one embodiment, the thalassemia is beta thalassemia.

In one embodiment of the first aspect, the inherited blood disorder is sickle cell anemia. In one embodiment, the sickle cell anemia is type HbSS. In one embodiment, the sickle cell anemia is type HbSC. In one embodiment, the sickle cell anemia is type HbS beta thalassemia. In one embodiment, the sickle cell anemia is type HbSD, HbSE, or HbSO.

In one embodiment of the first aspect or embodiments thereof, the BET inhibitor is JQ1.

In one embodiment of the first aspect or embodiments thereof, the BET inhibitor is CPI-0610.

In one embodiment of the first aspect or embodiments thereof, the BET inhibitor is PLX51107.

In one embodiment of the first aspect or embodiments thereof, the BET protein inhibitor inhibits bromodomain-containing protein 4 (BRD4).

In one embodiment of the first aspect or embodiments thereof, the BET protein inhibitor inhibits bromodomain-containing protein 2 (BRD2), bromodomain-containing protein 3 (BRD3), bromodomain-containing protein 4 (BRD4), and/or bromodomain testis-specific protein (BRDT).

In one embodiment of the first aspect or embodiments thereof, the BET inhibitor is administered to the subject orally.

In one embodiment of the first aspect or embodiments thereof, the BET inhibitor is administered to the subject intravenously.

In one embodiment of the first aspect or embodiments thereof, the BET inhibitor is administered to the subject in conjunction with a second therapy. In one embodiment, the second therapy comprises a histone deacetylase (HDAC) inhibitor. In one embodiment, the second therapy comprises an antineoplastic agent. In one embodiment, the antineoplastic agent comprises hydroxyurea. In one embodiment, the second therapy comprises an HbS polymerization inhibitor. In one embodiment, the HbS polymerization inhibitor is voxelotor. In one embodiment, the second therapy is a gene therapy approach. In one embodiment, the gene therapy approach comprises a lentiviral vector. In one embodiment, the gene therapy approach comprises gene editing.

In a second aspect, the present disclosure provides a method of inducing expression of fetal and/or embryonic hemoglobin in a subject, comprising administering a bromodomain and extra-terminal motif (BET) protein inhibitor to the subject.

In one embodiment of the second aspect, the fetal hemoglobin is hemoglobin F (HbF).

In one embodiment of the second aspect, the fetal hemoglobin is embryonic hemoglobin (HbE).

In one embodiment of the second aspect, the BET inhibitor is JQ1.

In one embodiment of the second aspect or embodiments thereof the BET inhibitor is CPI-0610.

In one embodiment of the second aspect or embodiments thereof, the BET inhibitor is PLX51107.

In one embodiment of the second aspect or embodiments thereof, the BET protein inhibitor inhibits bromodomain-containing protein 4 (BRD4).

In one embodiment of the second aspect or embodiments thereof, the BET protein inhibitor inhibits bromodomain-containing protein 2 (BRD2), bromodomain-containing protein 3 (BRD3), bromodomain-containing protein 4 (BRD4), and bromodomain testis-specific protein (BRDT).

In one embodiment of the second aspect or embodiments thereof, the BET inhibitor is administered to the subject orally.

In one embodiment of the second aspect or embodiments thereof, the BET inhibitor is administered to the subject intravenously.

In one embodiment of the first aspect, the therapeutically effective amount of the BET protein inhibitor induces expression of fetal and/or embryonic hemoglobin in the subject. In one embodiment, the expressed fetal and/or embryonic hemoglobin alleviates one or more symptoms of an inherited blood disorder in the subject. In one embodiment, the one or more symptoms of the inherited blood disorder comprises reduced clotting, excessive bleeding, fatigue, dizziness, malaise, anemia, joint pain, chest pain, delayed development, jaundice, or dactylitis.

In a third aspect, the present disclosure provides a method of inducing erythropoiesis in a subject, comprising administering to the subject a therapeutically effective amount of a bromodomain and extra-terminal motif (BET) protein inhibitor to the subject.

In one embodiment of the first, second, or third aspect, the BET inhibitor is one or more of JQ1, I-BET 151, I-BET 762, OTX-015, TEN-010, CPI-203, CPI-0610, olinone, RVX-208, LY294002, AZD5153, MT-1, MS645, RVX-297, PLX51107, BMS-986158, FT-1101, INCB054329, INCB057643, and ZEN-3694.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure. For all figures, *p<0.05, **p<0.01, ***p<0.001.

FIGS. 1A-1D. Effects of bromodomain and extra-terminal motif (BET) inhibitor JQ1 on cell growth and viability in erythroleukemia cell lines. Results for Days (D) 0, 1, 2, and 3 are shown. (1A) Cell growth of K562 cells with 200 nM JQ1 or dimethyl sulfoxide (DMSO) control. JQ1 slightly suppresses cell growth, but this effect was not statistically significant (n=3). (1B) Viability of K562 cells with 200 nM JQ1 or control. JQ1 did not affect cell viability in K562. (1C) Cell growth of HEL cells with 200 nM JQ1 or control. JQ1 significantly suppress cell growth by Day 3 (n=3). (1D) Viability of HEL cells with 200 nM JQ1 or control. JQ1 did not affect cell viability in HEL.

FIGS. 2A-2C. JQ1 treatment induces hemoglobin (HBG) expression (erythropoiesis) in K562 cells. Benzidine-hematoxylin staining was used to detect hemoglobin. Arrows indicate cells positive for HBG expression. (2A) Representative image of day 3 DMSO treated cells. (2B) Representative image of day 3 JQ1 treated cells. (2C) Quantification of percent positive for benzidine-hematoxylin (observed as red cells at Days (D) 3 and 5 of JQ1 treatment (white) versus DMSO control (black). N=3 for all data shown.

FIGS. 3A-3B. Effects of JQ1 on cell growth and viability in K562 cells. (3A) Cell growth of K562 cells with 200 nM JQ1 or control. JQ1 slightly suppress cell growth, but this effect was not statistically significant. (3B) Viability of K562 cells with 200 nM JQ1 or control. JQ1 did not affect cell viability in K562. N=3 for all data shown.

FIG. 4. RT-qPCR quantification of MYC expression in JQ1 (200 nM) or DMSO treated K562 cells. The DMSO control results are shown in black (diamonds); the JQ1 results are shown in grey (squares). Results for Days (D) 1, 2, 3, 4, and 5 are shown.

FIGS. 5A-5B. DMSO alone does not significantly change gene expression. (5A) Volcano plot of gene expression in day 0 vs. day 3 DMSO (N=2). (5B) Volcano plot of gene expression in day 0 vs. day 5 DMSO (N=2).

FIGS. 6A-6F. JQ1 induces erythropoiesis in K562 cells. (6A) Volcano plot of gene expression at day 3. JQ1 vs. DMSO treatment. (6B-6C) GO enrichment clusters of significantly upregulated (6B) and downregulated (6C) genes at day 3. (6D) Volcano plot of gene expression at day 5, JQ1 vs. DMSO treatment. (6E-6F) GO enrichment clusters of significantly upregulated (6E) and downregulated (6F) genes at day 5. N=2 for all data shown.

FIGS. 7A-7H. JQ1 treatment induces fetal and embryonic hemoglobin genes in erythroid cell lines. (7A-7C) RT-qPCR quantification of HBB (7A), HBG1/2 (7B), and HBE1 (7C) expression in K562 cells. (7D-7F) RT-qPCR quantification of HBB (7D), HBG1/2 (7E), and HBE1 (7F) expression in TF-1 cells. (7G) Percentage of HBB, HBG1/2, and HBE1 transcripts relative to total β-globin transcripts in K562 cells. (7H) Percentage of HBB, HBG1/2, and HBE1 transcripts relative to total β-globin transcripts in TF-1 cells. N=3 for all data shown.

FIGS. 8A-8D. Baseline gene expression in K562 cells (8A), HEL cells (8B), TF-1 cells (8C), and HL-60 (8D) cells as a percentage of 18S ribosomal RNA (log scale). MYC, HBB, HBG1/2, and HBE are shown.

FIGS. 9A-9F. JQ1 induces fetal and embryonic hemoglobin in erythroid, but not myeloid cell lines. (9A-9C) Expression of HBB (9A), HBG1/2 (9B), and HBE1 (9C) in HEL cells. (9D-9F) Expression of HBB (9D), HBG1/2 (9E), and HBE1 (9F) in HL-60 cells. N=3 for all data shown.

FIGS. 10A-10C. HBG1 and HBG2 expression was upregulated equally in response to JQ1. (10A) Differences in HBG1 and HBG2 sequences covered by the PCR amplicon. Stars indicate sites of base difference between the two genes. Three different sites of modified bases are shown: cytosine (C)/thymine (T) at Base 1, cytosine (C)/guanine (G) at Base 2, and thymine (T)/guanine (G) at Base 3. For the region surrounding and including Base 1, the sequence of HBG1 is TGCCACAAAGC (SEQ ID NO: 1), and the sequence of HBG2 is TGCCATAAAGC (SEQ ID NO: 2). For the region surrounding and including Base 2, the sequence of HBG1 is GACTGCAGTGG (SEQ ID NO: 3), and the sequence of HBG2 is GACTGGAGTGG (SEQ ID NO: 4). For the region surrounding and including Base 3, the sequence of HBG1 is ATGATTCAGAG (SEQ ID NO: 5), and the sequence of HBG2 is ATGATGCAGAG (SEQ ID NO: 6); (10B) Sequencing results showing PCR product from both HBG1 and HBG2 in K562. The ratio of the signal peak height was quantified and used as an approximation of expression ratios. (10C) Sequencing results showing PCR product from both HBG1 and HBG2 in HEL. Base 1 of HBG1 is evidently mutated from C to T, which made the two transcripts indistinguishable. The other two bases were used to quantify HBG1/HBG2 ratios.

FIGS. 11A-11D. JQ1 induces erythropoietic genes in K562 and TF-1 cells. (11A) Expression of alpha- and beta-cluster hemoglobin genes in K562 cells. Inset: HBD and HBG expression, adjusted y-axis. (11B) Expression of genes involved in heme biosynthesis in K562 cells. (11C) Expression of alpha- and beta-cluster hemoglobin genes in TF-1 cells. (11D) Expression of genes involved in heme biosynthesis in TF-1 cells. N=2 for all data shown.

FIGS. 12A-12L. Other BET inhibitors exhibited effects similar to JQ1 in erythroleukemia cell lines. (12A-12C) HBB (12A), HBG1/2 (12B), and HBE1 (12C) expression in K562 cells treated with CPI-0610. (12D-12F) HBB (12D), HBG1/2 (12E), and HBE1 (12F) expression in K562 cells treated with PLX51107. (12G-12I) HBB (12G), HBG1/2 (12H), and HBE1 (12I) expression in TF-1 cells treated with CPI-0610. (12J-12L) HBB (12J), HBG1/2 (18K), and HBE1 (18L) expression in TF-1 cells treated with PLX51107. N=3 for all data shown.

FIGS. 13A-13F. JQ1 downregulates known inhibitors of fetal hemoglobin. (13A) MYB expression in K562 and TF-1 cells. (13B) miR-15A expression in K562 and TF-1 cells. (13C) miR-16-1 expression in K562 and TF-1 cells. (13D) BCL11A expression in K562 and TF-1 cells. (13E) HEMGN expression in K562 and TF-1 cells. (13F) BCL11A expression in K562 and TF-1 cells. N=3 for all data shown.

FIGS. 14A-14N. Expression of known fetal hemoglobin regulators. (14A-14E) RNA-seq quantification of NR2F2, BCL11A, KLF1, GATA1, and HEMGN expression in K562 cells. (14F-14J) RNA-seq quantification of NR2F2, BCL11A, KLF1, GATA1, and HEMGN expression in TF-1 cells. (14K) RNA-seq quantification of MYB expression in K562 cells. (14L) RT-qPCR quantification of miR-15A and miR-16-1 in K562 cells. (14M) RNA-seq quantification of MYB expression in TF-1 cells. (14N) RT-qPCR quantification of miR-15A and miR-16-1 in TF-1 cells. N=3 for miR-15A and miR-16-1 (14L, 14N). N=2 for all other data shown.

FIGS. 15A-15C. JQ1 changes interaction frequencies between the LCR and the β-globin genes. (15A) 3C-qPCR quantification of interaction frequencies between the LCR and segments of the β-globin locus in DMSO vs. JQ1 treated K562 cells. (15B) 3C-qPCR quantification of interaction frequencies between the LCR and segments of the β-globin locus in DMSO vs. JQ1 treated TF-1 cells. (15C) 3C-qPCR quantification of interaction frequencies between the LCR and segments of the β-globin locus in EPO vs. EPO+JQ1 treated TF-1 cells. Track on top indicates the position of genes and the DNA fragments from EcoRI digestion. LCR: locus control region. HS: DNase hypersensitivity site. Anchor symbol indicate the fragment containing the bait primer (H-5432). N=3 for all data shown.

FIGS. 16A-16B. The LCR interaction with beta-globin genes was not changed by EPO treatment. 3C-qPCR quantification of the LCR interaction frequency with various sites in beta-globin cluster. N=3.

FIGS. 17A-17C. JQ1 changes interaction frequencies between BGLT3 and the fetal hemoglobin genes. (17A) 3C-qPCR quantification of interaction frequencies between BGLT3 and the fetal hemoglobin genes in DMSO vs. JQ1 treated K562 cells. (17B) 3C-qPCR quantification of interaction frequencies between BGLT3 and the fetal hemoglobin genes in DMSO vs. JQ1 treated TF-1 cells. (17C) 3C-qPCR quantification of interaction frequencies between BGLT3 and the fetal hemoglobin genes in EPO vs. EPO+JQ1 treated TF-1 cells. Track on top indicates the position of genes and the DNA fragments from EcoRI digestion. LCR: locus control region. HS: DNase hypersensitivity site. Anchor symbol indicate the fragment containing the bait primer (H-5432). N=3 for all data shown.

FIGS. 18A-18C. JQ1 does not affect GATA1 acetylation. (18A) 10% IP input from K562 nuclear lysate along with positive and negative controls for GATA1 antibody. TOP1 was used as loading control. Positive control: K562 nuclear lysate. Negative control: HEK-293T nuclear lysate. (18B) Anti-acetyl-lysine staining on immunoprecipretated K562 nuclear lysate. Normal rat-IgG was used for the IgG control. (18C) Total and acetyl-GATA1 quantification in DMSO or JQ1 treated K562 cells. Signal intensity was measured by ImageJ and normalized to corresponding TOP1 staining (N=3). n.s.: not significant.

### DETAILED DESCRIPTION

Provided herein are methods and compositions for treatment of sickle cell anemia and thalassemia.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term“about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to that amount of a therapeutic agent, such as a BET inhibitor, which when administered to a subject, is sufficient to effect treatment (e.g., improve symptoms) for a disease or disorder described herein, such as sickle cell disease. The amount of a compound which constitutes a “therapeutically effective amount” or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of the disease or disorder or one or more symptoms of the disease or disorder.

“Subject” refers to a warn blooded animal such as a mammal, preferably a human, which is afflicted with, or has the potential to be afflicted with one or more diseases and disorders described herein.

“Pharmaceutical composition” as used herein refers to a composition that includes one or more therapeutic agents disclosed herein, such as a BET protein inhibitor, a pharmaceutically acceptable carrier, a solvent, an adjuvant, and/or a diluent, or any combination thereof.

In view of the present disclosure, the methods and compositions described herein can be configured by the person of ordinary skill in the art to meet the desired need. In general, the disclosed materials and methods provide improvements in treating sickle cell anemia and thalassemia and similar blood disorders.

Inherited blood disorders manifest with a variety of symptoms. Some affect clotting (by reducing or effectively eliminated clotting) leading to easy bruising and/or excessive bleeding (e.g., frequent nosebleeds or excessive bleeding during or after invasive procedures). Hemophilia and von Willebrand Disease are examples of these clotting disorders. Other disorders affect the production or maintenance of red blood cells, and a number of these disorders affect hemoglobin. Additional symptoms associated with inherited blood disorders include fatigue, dizziness, malaise, anemia, joint pain, chest pain, delayed development, jaundice, and dactylitis.

Hemoglobin is an iron-rich protein in red blood cells. Oxygen entering the lungs attaches to the hemoglobin in the blood, which carries it to the tissues in the body. The oxygen is released from the hemoglobin in tissues throughout the body, allowing for aerobic respiration. When a subject has insufficient red blood cells or red blood cells that do not work properly, the body is left short of the oxygen it needs to function.

Human hemoglobin is a tetramer consists of two α-like globins and two β-like globins. The β-like globins are encoded by a cluster of genes located on chromosome 11p15.4 (HBB cluster). The HBB cluster consists of five genes: 5′-HBE1-HBG2-HBG1-HBD-HBB-3′. These genes are expressed during different stages of human development and undergo a series of epigenetic switches. HBE1 is expressed in the early embryonic stage and makes up the embryonic hemoglobins (Hb-Gower 1, ζ₂ε₂, and Hb-Gower 2, α₂ε₂) (1-3). HBG1 and HBG2 expression surpass HBE1 in the early fetal stage to make fetal hemoglobin (HbF, α₂γ₂), which is the predominant form of hemoglobin until months after birth. The switch from fetal to adult hemoglobin (HbA, α₂β₂) starts soon after birth, where HBB expression overtakes HBG1/2 to become the predominant form of hemoglobin (1,4). This switch does not completely shut down HbF production, and normal adults have HbF consisting ˜1% of all hemoglobin.

Expression of β-globin locus genes is under the control of locus control region (LCR), an enhancer-rich region 5′ of the gene cluster. Previous studies have shown that transcription factors (TFs) binding LCR promote looping and bring the enhancers in contact with promoters of β-globin genes (2). Forced looping of LCR to HBG1 and HBG2 leads to reactivation of HbF in adult CD34+ hematopoietic stem cells (HSCs) during erythropoiesis (5,6). In particular, the erythroid-lineage TFs GATA1 and KLF1 promote looping of LCR to the promoter of HBB (7-9). Notably, GATA1 requires the assistance of BET bromodomain proteins (e.g., BRD2, BRD3, BRD4, and BRDT) to bind to the β-globin locus (10,11).

The bromodomain (BRD) is a 110 amino acid protein domain that can recognize monoacetylated lysine residues. The recognition is a prerequisite for chromatin remodelling, and BRD-containing proteins play an important role in directing chromatin remodelling enzymes to specific sites. The BET family of proteins is characterized by the presence of two tandem bromodomains plus an extra-terminal (ET) domain. The ET domain is a conserved region of ˜80 amino acids that performs a regulatory function by recruiting specific effector proteins. The mammalian BET family of proteins comprises bromodomain-containing proteins 2, 3, and 4 (BRD2, BRD3, and BRD4) and bromodomain testis-specific protein (BRDT).

BET bromodomain inhibitors can cause reversion of the fetal-adult hemoglobin switch and increase the proportion of HbF in adult erythrocytes. Sickle cell anemia and thalassemia are genetic disorders that affect hemoglobin. There is a variety of subtypes of sickle cell anemia (also known as sickle cell disease, or SCD) with different genetic components. In the HbSS subtype, the subject inherits two sickle cell genes (“S”), one from each parent. This subtype is usually the most severe form of the disease. Subjects with HbSC inherit a sickle cell gene (“S”) from one parent and a gene for an abnormal hemoglobin called “C” from the other parent. This subtype is usually a milder form of SCD. Subjects who have HbS beta thalassemia inherit one sickle cell gene (“S”) from one parent and one gene for beta thalassemia from the other parent. There are two types of beta thalassemia: “0” and “+”. Those with HbS beta 0-thalassemia usually have a severe form of SCD. People with HbS beta +-thalassemia tend to have a milder form of SCD. There also are a few rarer types of SCD: HbSD, HbSE, and HbSO. People who have these forms of SCD inherit one sickle cell gene (“S”) and one gene for an abnormal type of hemoglobin (“D”, “E”, or “O”). The severity of these rarer types of SCD varies.

In individuals who have sickle cell trait (SCT), or HbAS, one sickle cell gene (“S”) is inherited from one parent and one normal gene (“A”) from the other parent. People with SCT usually do not have any of the signs of the disease and live a normal life, but they can pass the trait on to their children. Additionally, there are a few uncommon health problems that may be related to sickle cell trait.

Thalassemia has two main types: alpha thalassemia and beta thalassemia. In alpha thalassemia, the hemoglobin does not contain enough alpha protein. To make alpha-globin protein chains, four genes are needed: two on each chromosome 16. If one or more of these genes is missing, alpha thalassemia will result. Two globin genes are needed to make beta-globin chains—one on each chromosome 11. If one or both genes are faulty, beta thalassemia will occur. The severity of thalassemia in each case depends on how many genes are faulty, or mutated. The words “trait,” “minor,” “intermedia,” or “major” are used to describe the severity of thalassemia. For example, a person who has thalassemia trait may not have any symptoms at all or may have only mild anemia, while a person with thalassemia major may have severe symptoms and may need regular blood transfusions.

Based on the present disclosure, it is believed that inhibitors of bromodomain and extra-terminal motif (BET) proteins (referred to as “BET protein inhibitors” or “BET inhibitors”) can be effective for treating different subtypes of sickle cell disease and various types and severities of thalassemia. While not wishing to be bound by theory, it is believed that BET protein inhibitors can be used to induce and/or enhance the expression of normal fetal and/or embryonic hemoglobin genes in individuals having mutated adult hemoglobin genes to alleviate symptoms of different subtypes of sickle cell disease and/or various types and severities of thalassemia.

Inhibitors of BET proteins prevent the protein-protein interactions between BET proteins and acetylated histones and transcription factors. A BET inhibitor can modulate activity of one or more of BRD2, BRD3, BRD4, and BRDT. Non-limiting examples of BET inhibitors contemplated for use in the present disclosure include one or more of JQ1 (a thienotriazolodiazepine; available from Sigma-Aldrich), I-BET 151 (GlaxoSmithKline), I-BET 762 (also known as GSK525762, molibresib; GlaxoSmithKline), OTX-015 (also known as MK-8628; Merck Sharp & Dohme Corporation), TEN-010 also known as RO6870810 (Hoffman La-Roche), CPI-203 (Constellation Pharmaceuticals), CPI-0610 (Constellation Pharmaceuticals), olinone, RVX-208 (also known as RVX000222, apabetalone; Resverlogix Corporation), LY294002, AZD5153 (AstraZeneca), MT-1, MS645, and RVX-297 (Resverlogix Corporation), PLX51107 (Plexxicon), BMS-986158 (Bristol-Myers Squibb), FT-1101 (Forma Therapeutics), INCB054329 (InCyte Corporation), INCB057643 (InCyte Corporation), ZEN-3694 (Zenith Epigenetics), and combinations thereof.

Compositions

In some embodiments, pharmaceutical compositions contemplated herein include a therapeutically effective amount of one or more BET inhibitors. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

BET inhibitors of the present disclosure can be administered through a variety of routes and in various compositions. For example, compositions containing BET inhibitors can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, one or more BET inhibitors is administered orally, and the subject is dosed on a daily basis.

The therapeutic agents (also referred to as “compounds” herein) described herein (e.g., BET inhibitors and secondary therapeutic agents), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Non-limiting examples of contemplated secondary therapeutic agents include one or more histone deacetylase (HDAC) inhibitors. Examples of histone deacetylase inhibitors include panobinostat, entinostat, romidepsin, and vorinostat. Other therapies can comprise an antineoplastic agent. In some embodiments, the antineoplastic agent is hydroxyurea. Other therapies can also include an HbS polymerization inhibitor. In some embodiments, the HbS polymerization inhibitor is voxelotor. Other therapies can include administration of a hematopoietic stem cell mobilizer. The stem cell mobilizer can be plerixafor.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of BET inhibitors and secondary therapeutic agents can be in the range of from about 0.0001 mg/kg/day, about 0.001 mg/kg/day, or about 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 ml of a pharmaceutical composition having a concentration of a BET inhibitor at about 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 1000 μM in a pharmaceutically acceptable carrier.

In some embodiments, the subject is tested before treatment to determine which BET inhibitor will be most effective. This testing can be performed by isolating primary hematopoietic stem cells from the subject and performing an in vitro drug screening. The BET inhibitor found to be most effective in the isolated hematopoietic stem cells of the subject, for example, resulting in the most desirable gene expression in the cells, can be used to treat the subject.

Methods

In some embodiments, methods of treating an inherited blood disorder in a subject in need thereof include administering to the subject a therapeutically effective amount of one or more bromodomain and extra-terminal motif (BET) protein inhibitors and optionally a second therapy and/or secondary therapeutic agent. Treatable inherited blood disorders can include thalassemias (alpha thalassemia and beta thalassemia) and sickle cell anemia (type HbSS, HbSC, HbS beta thalassemia, HbSD, HbSE, or HbSO.

In some embodiments, the therapeutic methods contemplated herein include inhibiting bromodomain-containing protein 4 (BRD4), bromodomain-containing protein 2 (BRD2), bromodomain-containing protein 3 (BRD3), bromodomain-containing protein 4 (BRD4), and/or bromodomain testis-specific protein (BRDT).

In some embodiments, the therapeutic methods contemplated herein include administering to the subject a pharmaceutical composition to the subject orally and/or intravenously.

In some embodiments, the therapeutic methods contemplated herein include administering to the subject a pharmaceutical composition including both one or more BET protein inhibitors and one or more secondary therapeutic agents. In other embodiments, the therapeutic methods include administering a first pharmaceutical composition including one or more BET protein inhibitors and a second pharmaceutical composition including one or more secondary therapeutic agents.

In some embodiments, the therapeutic methods contemplated herein include administering to the subject a pharmaceutical composition including one or more BET protein inhibitors and administering a gene therapy approach to treat the inherited blood disorder in the subject. For example, the gene therapy approach can be administration of a lentiviral vector to the subject and/or ex vivo gene editing of hematopoietic tissues taken from the subject.

In some embodiments, the therapeutic methods contemplated herein include inducing expression of fetal and/or embryonic hemoglobin in a subject by administering a bromodomain and extra-terminal motif (BET) protein inhibitor to the subject. For example, the hemoglobin can be fetal hemoglobin F (HbF) and/or embryonic hemoglobin (HbE) and the expressed fetal and/or embryonic hemoglobin can alleviate one or more symptoms of an inherited blood disorder in the subject. In some embodiments, the one or more symptoms of the inherited blood disorder comprises reduced clotting, excessive bleeding, fatigue, dizziness, malaise, anemia, joint pain, chest pain, delayed development, jaundice, or dactylitis.

In some embodiments, the therapeutic methods contemplated herein include a method of inducing erythropoiesis in a subject including administering to the subject a therapeutically effective amount of a bromodomain and extra-terminal motif (BET) protein inhibitor.

In some embodiments, the therapeutic methods contemplated herein include treating a subject having or suspected of having a hemoglobin-related disorder, such as sickle cell anemia, thalassemia, and similar blood disorders, with a therapeutic amount of one or more BET inhibitors including JQ1, I-BET 151, I-BET 762, OTX-015, TEN-010, CPI-203, CPI-0610, olinone, RVX-208, LY294002, AZD5153, MT-1, MS645, RVX-297, PLX51107, BMS-986158, FT-1101, INCB054329, INCB057643, and ZEN-3694.

In some embodiments, a therapeutic method contemplated herein includes treating a subject having an abnormal hemoglobin by obtaining a sample from the subject (e.g., blood, plasma, urine, saliva, or tissue), detecting an abnormal hemoglobin in the sample, and administering a therapeutic amount of one or more BET inhibitors and/or a secondary therapeutic agent to the subject. In this context, the term “abnormal hemoglobin” refers to any abnormality detected in the subject's hemoglobin such as those that adversely affect hemoglobin structure or hemoglobin function, abnormal hemoglobin expression levels, abnormal hemoglobin clearance rates, abnormal types of hemoglobin expressed, and the like.

In some embodiments of the present disclosure, the response of a subject to BET inhibitor administration is used to diagnose an HBB-related disorder. For example, a method of treating a subject for an HBB-related disorder can include administering a therapeutically effective amount of a BET inhibitor to a subject suspected of having HBB-related disorder, and alleviating one or more symptoms associated with the HBB-related disorder, wherein the alleviation of the one or more symptoms by administration of the BET inhibitor is indicative of the subject having an inherited blood disorder.

In some embodiments, one or more BET inhibitors can be administered in conjunction with another therapy or therapies for an inherited blood disorder (a second therapy or secondary therapeutic agent). In some embodiments, the BET inhibitor is delivered concurrently with the other therapy or therapies or administration can be in series (e.g., a BET inhibitor is administered before or after a secondary therapeutic agent).

In some embodiments, the BET inhibitor treatment is accompanied by a gene therapy approach. The gene therapy can be performed ex vivo, and the subject can be treated with autologous hematopoietic cells. The gene therapy approaches can function, for example, by adding a cassette to drive expression of a normal beta chain. The gene therapy approach can comprise a lentiviral therapy. The lentiviral vector can comprise, for example, a human β-A-T87Q globin gene (LentiGlobin BB305, bluebird bio).

Alternatively or in addition, the gene therapy approach can function through gene editing. For example, the gene therapy approach can use clustered regularly interspaced short palindromic repeats (CRISPR) and CRISPR associated protein 9 (Cas9), zinc finger nuclease, or related technologies to correct one or more defective genes, or alter expression of compensatory genes. For example, the gene therapy approach can involve engineering hematopoietic stem cells to produce high levels of fetal hemoglobin by editing the BCL11A gene (CTX001, Vertex/CRISPR Therapeutics; BIVV003, Bioverativ/Sangamo).

### EXAMPLES

The Examples that follow are illustrative of specific embodiments of the disclosure, and various uses thereof. They are set forth for explanatory purposes only and should not be construed as limiting the scope of the disclosure in any way.

### Example 1: BET Bromodomain Inhibitors Enhance the Expression of Fetal and Embryonic Hemoglobin Genes

### Summary

The human hemoglobin genes consist of three distinct types of hemoglobin expressed at different developmental stages. The embryonic hemoglobin is produced at the earliest stages of embryonic development and is soon replaced by fetal hemoglobin (HbF). HbF persists throughout prenatal development and is overtaken by the production of adult hemoglobin (HbA) after a few months after birth. The mechanisms resulting in the fetal to adult hemoglobin switch at the β-globin locus involves numerous protein complexes and relies on proper chromatin looping. In inherited blood disorders that involve mutations in the adult hemoglobin gene, such as sickle cell anemia (SCA), a major treatment strategy is to reactivate fetal hemoglobin to partially replace the mutated sickle hemoglobin (HbS). However, options for pharmacological inducers of fetal hemoglobin remain few beyond hydroxyurea. Here, it is reported that BET inhibitors induced specific upregulation of fetal and embryonic hemoglobin production in vitro. In cells expressing the adult hemoglobin gene HBB, JQ1 treatment reduces the proportion of adult hemoglobin transcripts in favor of fetal and embryonic hemoglobin genes. Mechanistically, JQ1 treatment reduces the expression of fetal hemoglobin inhibitors MYB and BCL11A. In TF-1 cells, JQ1 also upregulates the lncRNA gene BGLT3, which leads to increased enhancer contact with the HbF genes. These results suggest that small molecule BET inhibitors could have clinical utility in the treatment of SCA and other inherited hemoglobinopathies by upregulating HbF and embryonic hemoglobin genes.

### Introduction

Previous studies have shown that BET inhibitors induce differentiation of erythroid cell lines UT7, but they did not characterize which hemoglobin genes were expressed (12). Although some studies have performed RNA-seq on JQ1-treated K562 cells, none has focused on the differentiation properties of JQ1, and the length of the treatment was generally too short to characterize erythropoietic phenotypes (13-16). Here, how BET inhibitors affect the interaction between the LCR and its target hemoglobin genes and the expression of genes regulating erythropoiesis was examined. It was hypothesized that BET inhibitors would promote expression of erythropoietic genes and suppress genes involved in myeloid differentiation. In addition, since BET proteins are heavily involved in the looping between LCR and the adult hemoglobin genes, it was reasoned that BET bromodomain inhibitors would disrupt this interaction and increase the expression of fetal and embryonic hemoglobins, suggesting likely clinical utility.

### Materials and Methods

Cell Lines and BET Inhibitor Treatments

K562, HEL, and HL-60 cells were cultured in RPMI 1640 medium supplemented with 10% FBS. TF-1 cells were cultured in RPMI 1640 medium supplemented with 10% FBS and 4 ng/mL GM-CSF. Before treatment, TF-1 cells were washed 4× with RPMI 1640 without supplements, and cultured in RPMI 1640 medium with 10% FBS but no GM-CSF for 24 hours. 4 ng/mL GM-CSF or 3 units/mL EPO were added at the time of treatment set up. K562, HEL, and HL-60 cells were cultured as normal until treatment set up. JQ1 (Sigma-Aldrich, SML1524), CPI-0610 (Selleck Chemicals, S7853), and PLX51107 (MedChemExpress, HY-111422) were dissolved in DMSO to 10 mM concentration and stored in −80° C. All three inhibitors were applied to cells at 200 nM, with parallel DMSO treatment (0.002%) as control. JQ1 and PLX51107 were added to cell culture at day 0 without additional treatments up to day 5. CPI-0610 was reapplied to the cells on a daily basis.

Cytospin and Benzidine-Hematoxylin Staining

K562 cells were counted, and 200,000 cells were collected by spinning at 400 g. Culture media was discarded, and cells were re-suspended in 100 μL of PBS. The cells were spun onto glass slides at 600 rpm, which were allowed to dry for ˜20 minutes before staining. The slides were stained in 0.2% benzidine solution in 0.5 M acetic acid for 4 minutes, followed by 2 minutes in 2% H₂O₂ in 70% ethanol, and 3 minutes washing in running tap water. After the wash, the slides were further stained in 1× hematoxylin solution for 1 minute, followed by another 3 minutes wash in running tap water. The slides were then air-dried and mounted.

Microscopy and Quantification of Benzidine Staining

At least three randomly selected fields of stained cells were imaged at 40× magnification. Images were processed using the Color Deconvolution plugin in ImageJ software to separate hematoxylin and benzidine staining to different channels (37). The numbers of benzidine-positive cells and the intensity of benzidine staining were quantified in the benzidine channel, whereas the total cell number and abnormal nuclear morphology were quantified in the hematoxylin channel. Results of all fields from each slide were summed for the final quantification.

qPCR Quantification of Gene Expression

cDNA libraries for mRNAs were made from 1 μg input RNA using High-Capacity cDNA Reverse Transcription Kit (ThermoFisher, 4368814). Gene expressions were quantified using Power SYBR Green PCR Master Mix (Thermofisher, 4367659). Primer sequences used in qPCR are listed in Table 1. For microRNAs, cDNA libraries were made with Taqman Advanced miRNA cDNA Synthesis Kit (Thermofisher, A28007). miR-15A and miR-16-1 were quantified using the Taqman Advanced miRNA Assays (ThermoFisher, A25576) specific to hsa-miR-15a-5p (assay ID: 477858_mir) and hsa-miR-16-5p (assay ID: 477860_mir).

RNA-seq and Data Processing

Sample RNA libraries were prepared using the KAPA mRNA HyperPrep Kit (Roche, KK8580) with 1 μg input RNA and submitted for sequencing. Raw reads in fastq format were aligned to the hg19 reference genome using HISAT2(38) with Gencode gene and transcript annotation (GRCh37.p13)(39). Gene expression was quantified and compared by tools in the Cuffliks package (40). GO analysis and term clustering was performed using g:Profiler and Cytoscape following the protocols published by Reimand et al (41).

3C-qPCR Quantification of LCR Interaction Frequency

Methods for chromatin conformation capture (3C) qPCR was adapted from Hagège et al. (42). Briefly, 10 million cells were collected and fixed with 1% formaldehyde for 5 minutes in PBS with 10% FBS for 5 minutes, then quenched by 124 mM glycine on ice. The fixed cells were then lysed and digested by 400 units of EcoRI (NEB, R3101). Small aliquots of the samples were taken before and after the digestion as digestion controls. Following the digestion, the samples were diluted and ligated by T4 DNA ligase (NEB, M0202). The samples were then reverse-crosslinked, and DNA was purified by phenol-chloroform extraction. The digestion efficiency, sample purity, and DNA concentration of each sample were assessed by qPCR to exclude low-quality samples prior to the 3C quantification. Previously published 3C primers were used (43,44), listed in Table 1. Primer pairings for all processes in 3C-qPCR are listed in Table 2. Calculated quantity of each primer pairs was normalized as number of templates per million B13 control site template. Additionally, because the Gg primer anneals to both HBG1 and HBG2 fragments, while the Ag primer anneals to only HBG2 fragment, the Gg primer signal was subtracted by Ag primer signal to quantify the HBG1 interaction frequency. All 3C-qPCR was performed using Power SYBR Green PCR Master Mix (ThermoFisher, 4367659).

Immunoprecipitation (IP) and Quantification of Acetyl-GATA1

Nuclear protein was extracted according to previously described protocol (45). 10% of the nuclear protein extraction was saved as input. The remaining nuclear extract was diluted with non-denaturing IP buffer (20 mM Tris-HCl pH 8, 137 mM NaCl, 10% glycerol, 1% Triton X-100, 2 mM EDTA) and pre-cleared with normal rat IgG (Santa Cruz, sc2026) and Dynabeads Protein G (ThermoFisher, 10004D). Pre-cleared solutions were split into two equal portions, and 5 μg of anti-GATA1 antibody (Santa Cruz, sc266) or normal rat IgG were added to either portion and incubated overnight at 4° C. overnight. Antibodies were then pulled-down using Dynabeads Protein G, and washed 1× with the IP buffer and 2× with PBS containing 1% Triton X-100. The beads were boiled in 2× protein loading buffer to dissociate the proteins. Input and IP-ed proteins were ran on SDS-PAGE gels and transferred to PVDF membranes. The input samples were blotted using anti-topoisomerase 1 (Abcam, ab109374) and anti-GATA1 antibodies, and the IP-ed samples were blotted with anti-acetyl-lysine antibody (Abcam, ab21623). Quantification of signal intensity was done by ImageJ, and GATA1 and acetyl-GATA1 signals were normalized to the topoisomerase 1 signals in each sample.

### Results

BET Inhibitor JQ1 Upregulates Fetal and Embryonic Hemoglobin in K562 Cells

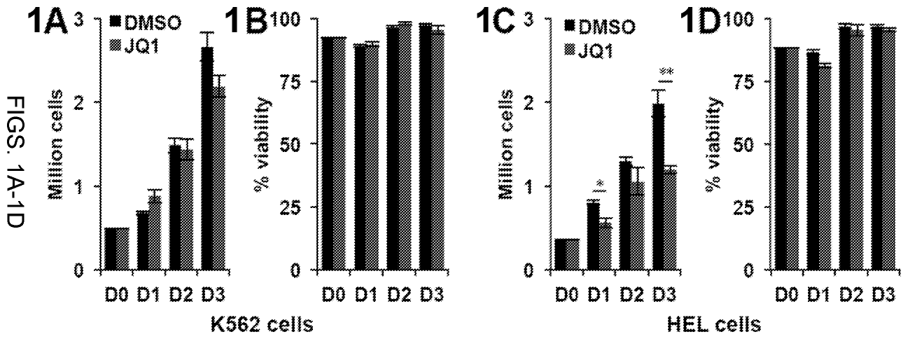

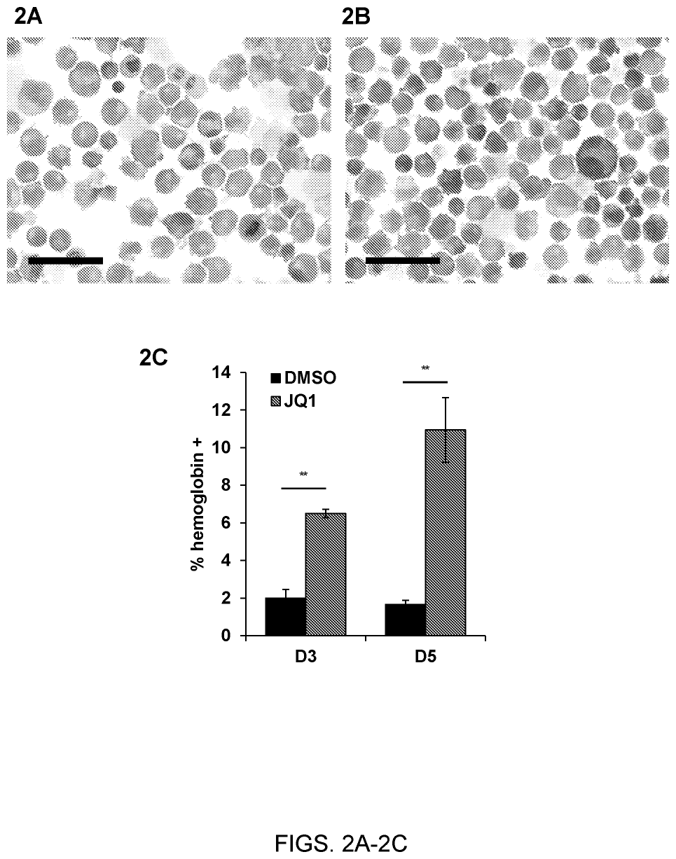

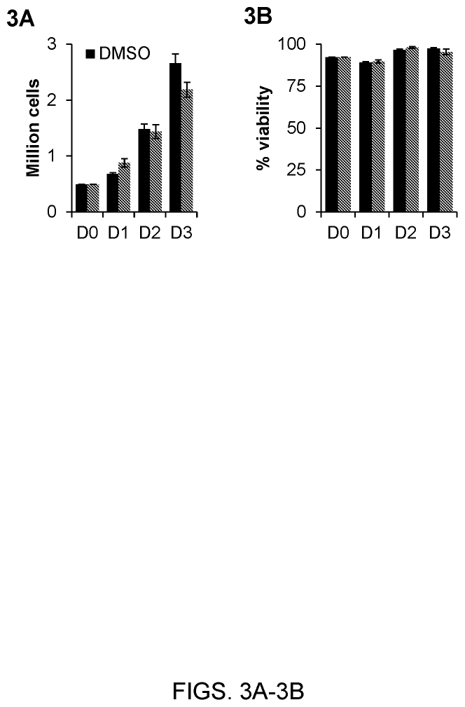

BET inhibitors are known to suppress MYC expression. To test whether MYC inhibition leads to enhanced erythroid gene expression, erythroleukemia cell lines were treated with 200 nM JQ1 or DMSO control and incubated for up to 5 days. JQ1 treatment caused K562 and HEL cells to grow slower, but cell viability was unaffected in either cell line (FIGS. 1A-1D). The percentage of cells that accumulated hemoglobin by benzidine-hematoxylin staining were quantified (FIGS. 2A-2C). By day 3, JQ1 treated K562 cells (FIG. 2B) have more hemoglobin producing cells (˜6%) compared to control (˜2%, FIG. 2A). By day 5, over 10% of JQ1 treated K562 cells were producing hemoglobin (FIG. 2C). Cell growth and viability were not significantly affected by the JQ1 treatment (FIGS. 3A-3B).

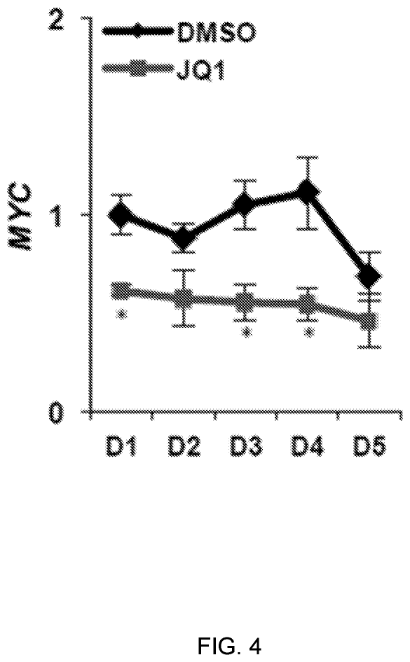

Quantitative real-time PCR (qPCR) showed that MYC expression was consistently suppressed up to 4 days after the initial treatment (FIG. 4).

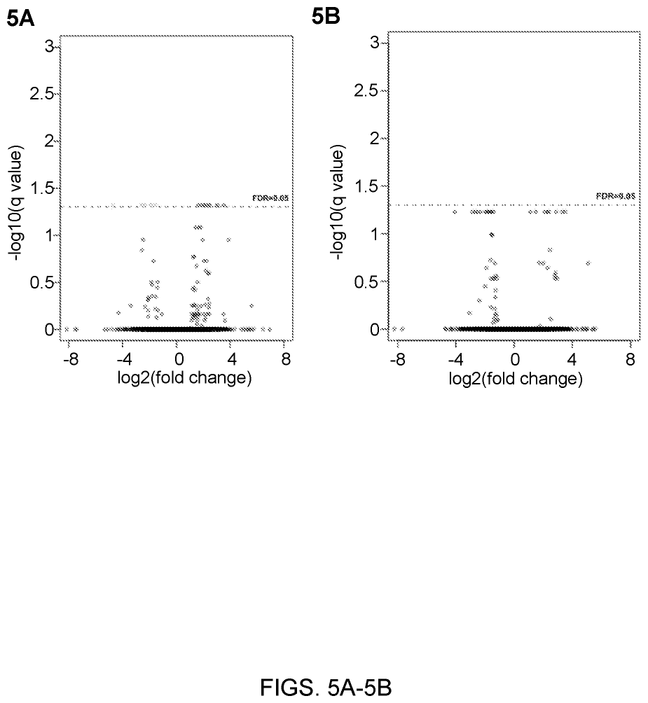

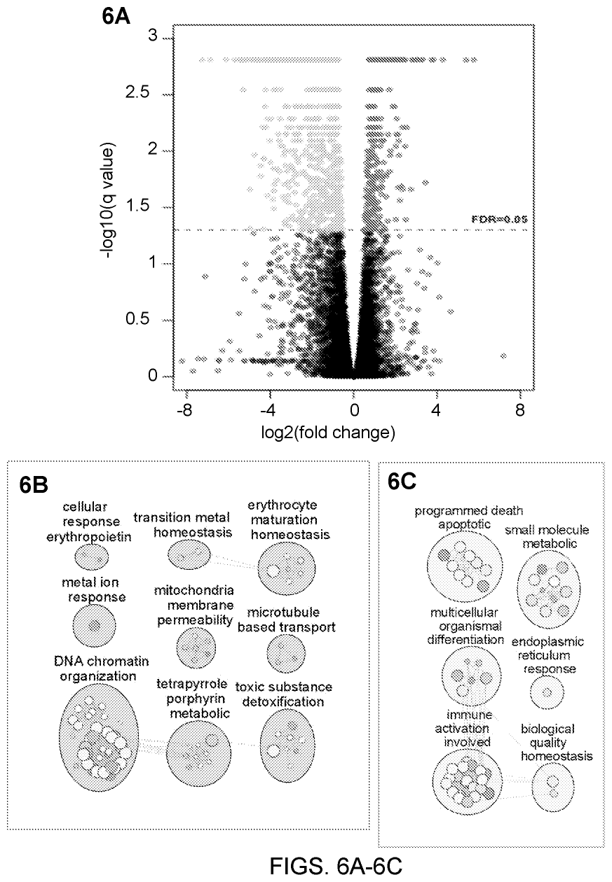

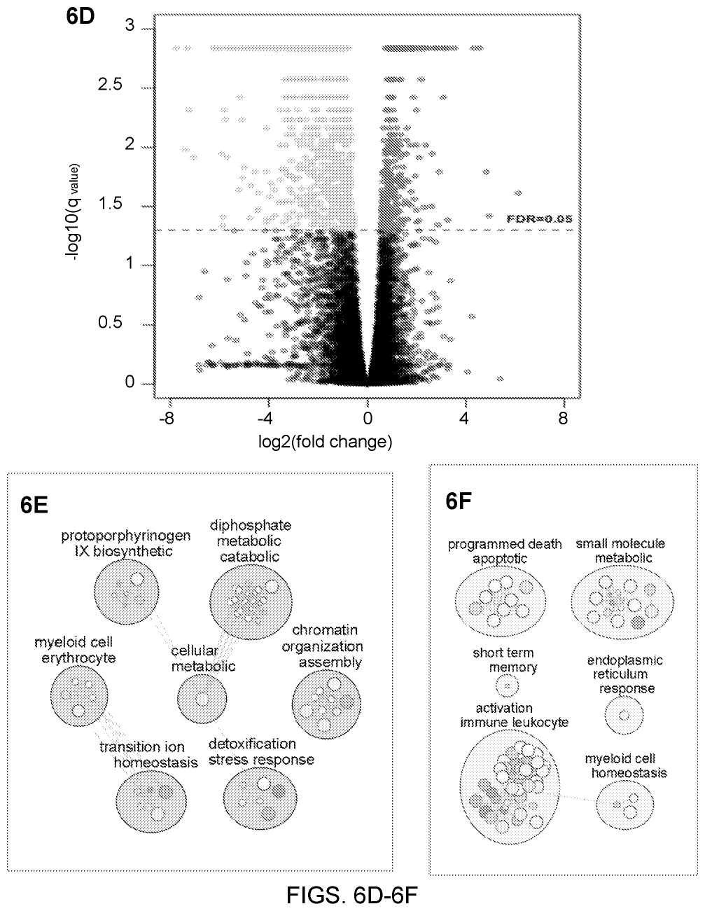

To define the transcriptomic changes that accompany erythropoiesis, RNA-sequencing (RNA-seq) was performed throughout erythroid differentiation. Although DMSO alone did not induce changes in gene expression (FIGS. 5A-5B), JQ1 substantially changed the transcriptome on both day 3 and day 5 (FIG. 6A, 6D). Gene ontology (GO) analysis was performed on up- or down-regulated genes at both time points, followed by clustering of the GO terms by similarity. Labeling of each cluster was automatically generated by displaying the top 3 common words from all GO terms within the cluster. Notably, multiple clusters of upregulated GO terms at day 3 (FIG. 6B) are related to erythropoiesis, including “cellular response erythropoietin”, “transition metal homeostasis” (iron transport), “erythrocyte maturation homeostasis”, and “tetrapyrrole porphyrin metabolic” (heme biosynthesis). In contrast, the downregulated GO terms at day 3 included the cluster “immune activation involved” (FIG. 16C), which consists of many genes involved in myeloid differentiation. Similarly, day 5 upregulated clusters included “protoporphyrinogen IX biosynthesis” (heme biosynthesis), “myeloid cell erythrocyte”, and “transition ion homeostasis” (iron transport) (FIG. 6E), whereas the downregulated cluster included “activation immune leukocyte” (FIG. 6F). These results indicate that JQ1 specifically promotes erythroid, but not myeloid differentiation in K562 cells.

BET Inhibitors Induce Fetal and Embryonic, but not Adult Hemoglobin

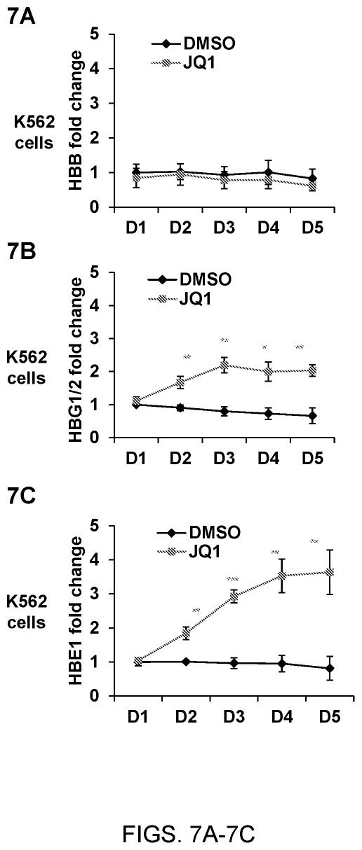

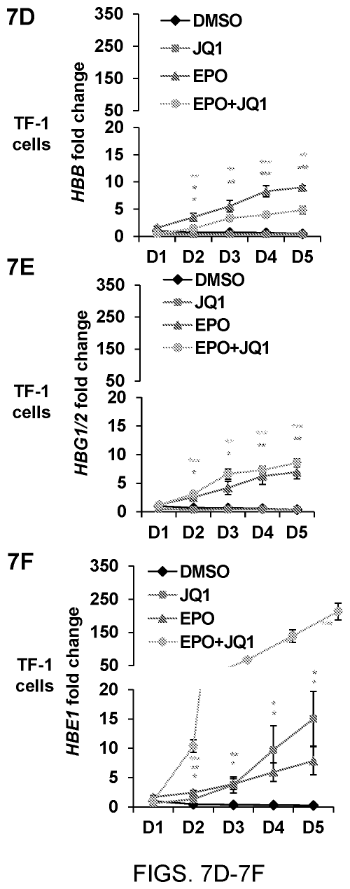

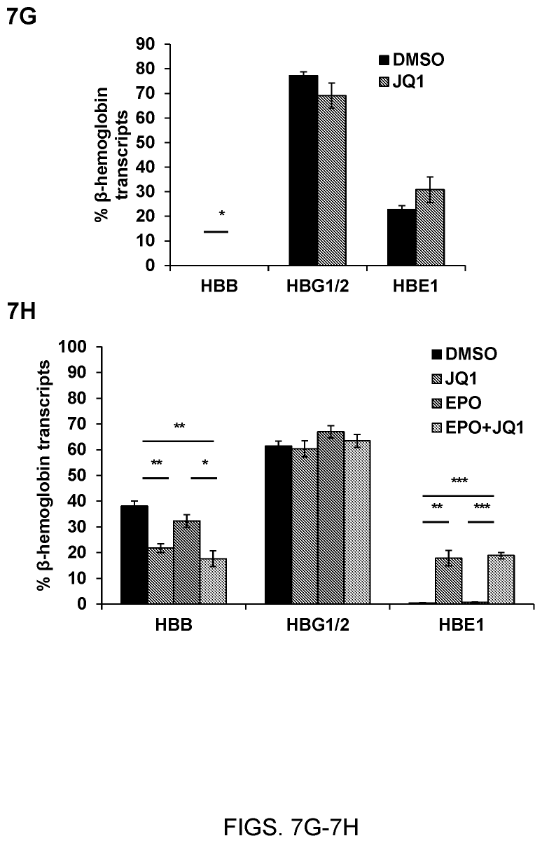

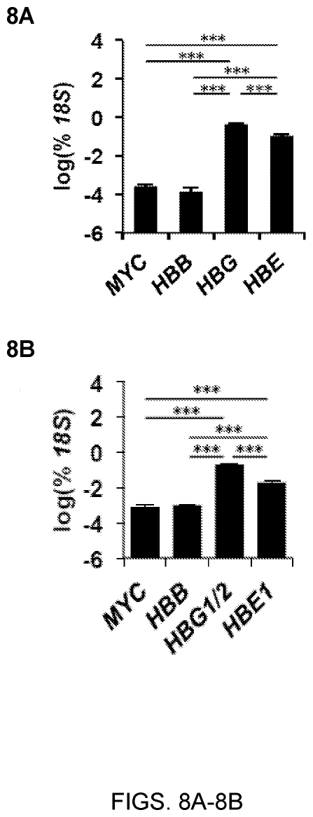

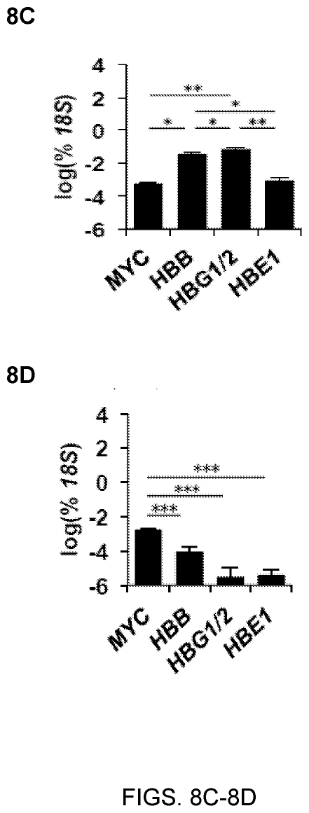

To characterize the types of hemoglobin genes upregulated by JQ1, RT-qPCR primers were designed to quantify the expression of HBB, HBG1/2, and HBE1 in a 5-day time course. Only HBG1/2 and HBE1 were upregulated by JQ1, whereas HBB expression was surprisingly unaffected (FIGS. 7A-7C). However, the base expression of adult hemoglobin genes in K562 cells is very low compared to HBE1 and HBG1/2 (FIG. 7G), therefore it is possible that the effects of JQ1 on HBB and HBD were undetectable. To address this issue, TF-1 cells were treated with JQ1. TF-1 cells require the cytokine GM-CSF to survive (proliferative condition). These cells can also undergo erythroid differentiation when stripped of GM-CSF and provided with erythropoietin (EPO) (erythropoietic condition). In TF-1 cells, EPO stimulation led to upregulation of all three types of hemoglobin, whereas JQ1 treatment only upregulated the embryonic hemoglobin HBE1 (FIGS. 7D-7F). Strikingly, when TF-1 cells were treated with both EPO and JQ1, HBE1 expression increased over 200-fold by day 5, and HBB expression was significantly decreased (FIGS. 7D-7F). Together, under both proliferation and erythropoietic conditions. JQ1 treatment significantly decreased the proportion of HBB transcripts and increased HBE1 transcripts (FIG. 7H). This treatment was repeated on two other cell lines, HEL and HL-60. HEL is an erythroid leukemia cell line similar to K562 (having a low HBB base expression (FIGS. 8A-8D)), whereas HL-60 is a promyelocytic leukemia cell line. JQ1 significantly upregulated both HBG1/2 and HBE1 while suppressing HBB in HEL cells (FIGS. 9A-9C), but had no effect on these genes in HL-60 cells (FIGS. 9D-9F), suggesting that this effect is erythroid specific.

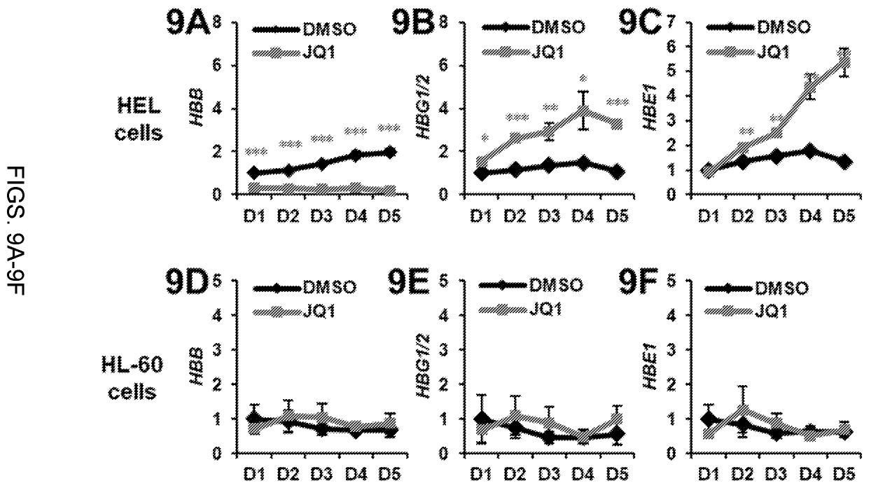

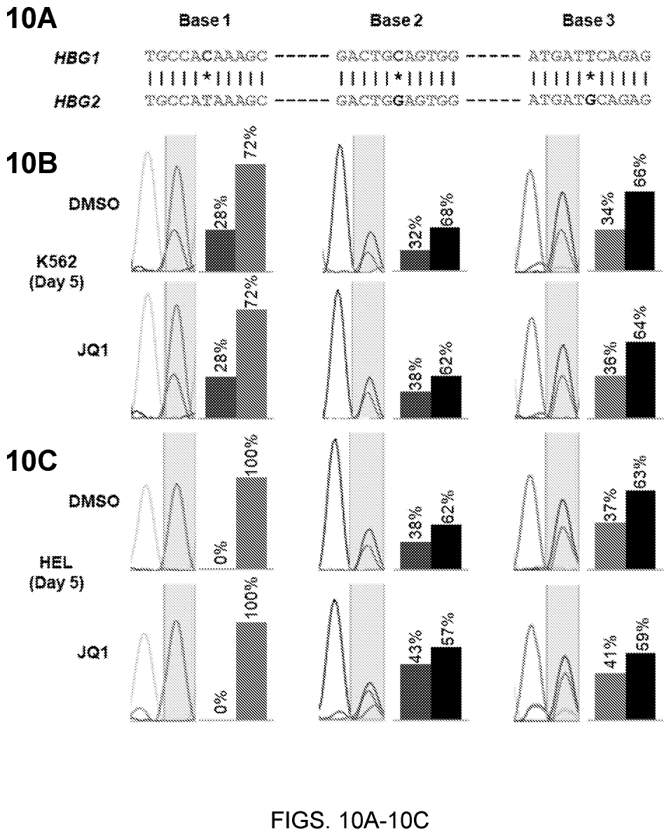

Next, it was determined whether HBG1 and HBG2 were differentially upregulated in K562 and HEL cells. Because the HBG1/2 primers could not distinguish between the two fetal hemoglobin genes, a segment of the HBG1/2 cDNA containing 3 differential bases was amplified and Sanger sequencing was used to measure the difference in signal intensity at each of these base (FIG. 10A) By measuring the peak heights of the signals corresponding to either HBG1 or HBG2, no significant changes in the ratio of HBG1 vs. HBG2 comparing DMSO vs JQ1 treatment were observed (FIGS. 10B and 10C). This was confirmed by RNA-seq data of K562 cells (FIG. 11A).

RNA-seq data were next examined for both the K562 and TF-1 cells to validate the qPCR results (FIGS. 11A-11D). In K562 cells, JQ1 treatment increased the expression of all α- and β-globin hemoglobin genes except HBB and HBD (FIG. 11A). JQ1 also upregulated multiple genes encoding enzymes in the heme biosynthesis pathway, notably ALAS2, which encodes the rate-limiting enzyme 5′-Aminolevulinate Synthase 2 (FIG. 11B). In TF-1, JQ1 specifically induced the embryonic hemoglobin gene HBE1 in the β-globin locus, but upregulated the adult hemoglobin genes HBA1/2 in the α-globin locus (FIG. 11C). Compared to the selective upregulation by JQ1, EPO treatment led to upregulation of all hemoglobin genes (FIG. 10C). Similar to that in K562 cells, ALAS2 was upregulated by JQ1 in TF-1 cells, especially under erythropoietic conditions (FIG. 11D).

Other BET Inhibitors Showed Similar Effects in Erythroleukemia Cell Lines

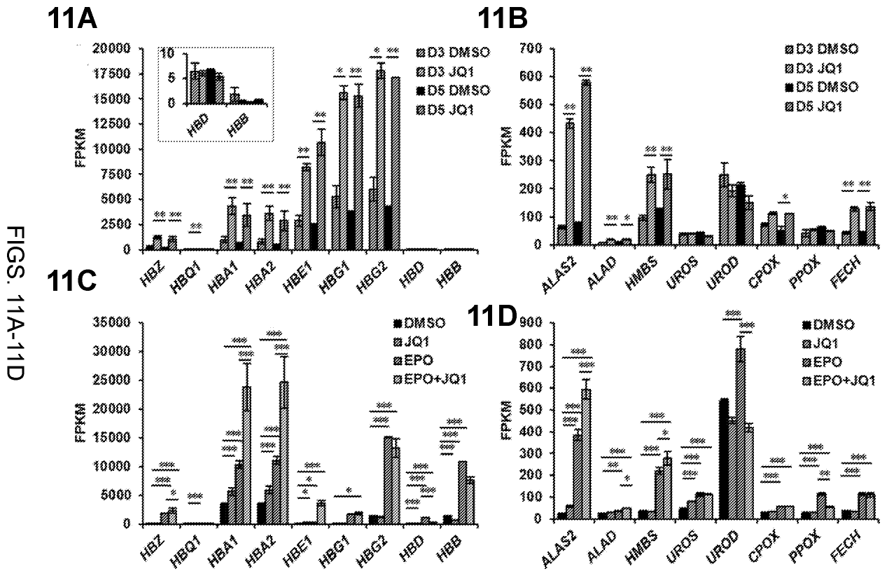

To ensure that the upregulation of fetal/embryonic hemoglobin observed is not unique to JQ1, two additional BET inhibitors, CPI-0610 and PLX51107, were tested. Unlike JQ1, which has a higher affinity for BRD3 and BRD4 (17), CPI-0610 binds to BRD3, BRD3, BRD4, and BRDT with comparable affinity with a preference for the second bromodomain (BD2) of the BET proteins (18). In contrast, PLX51107 preferentially binds to BD1 and has a somewhat lower affinity for BRDT. CPI-0610 is currently in phase II clinical trials for those with myelofibrosis or myelodysplastic syndrome (MDS) in combination with ruxolitinib. PLX51107 is currently in phase I clinical trials for individuals with acute myeloid leukemia or MDS in combination with azacitidine. Erythroid cell lines were treated with these drugs to measure their effects on the expression of adult, fetal, or embryonic hemoglobin genes. CPI-0610 has a short lifespan in culture medium, and was reapplied daily, whereas PLX51107 was added once at day 0 without additional treatment. Both CPI-0610 and PLX51107 were observed to mimic the effects of JQ1 in K562 and TF-1 cells (FIGS. 12A-12L). In particular, PLX51107 significantly suppressed HBB expression in TF-1 cells under proliferative condition (FIG. 12J), which was not observed under JQ1 treatment. These results suggest that BET inhibitors in general have the potential to specifically upregulate fetal and embryonic hemoglobin genes.

JQ1 Downregulates Known Inhibitors of Fetal Hemoglobin

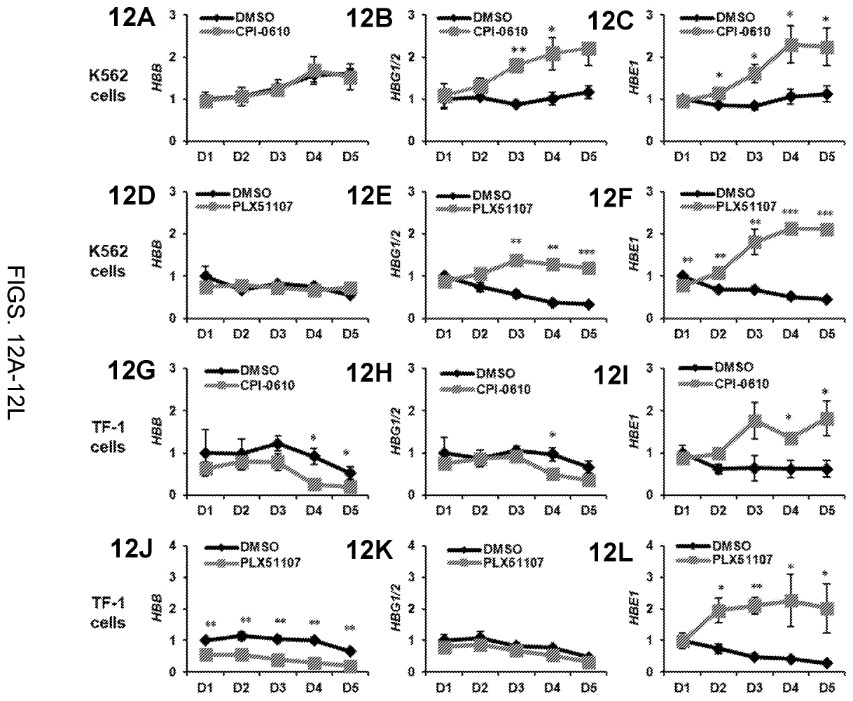

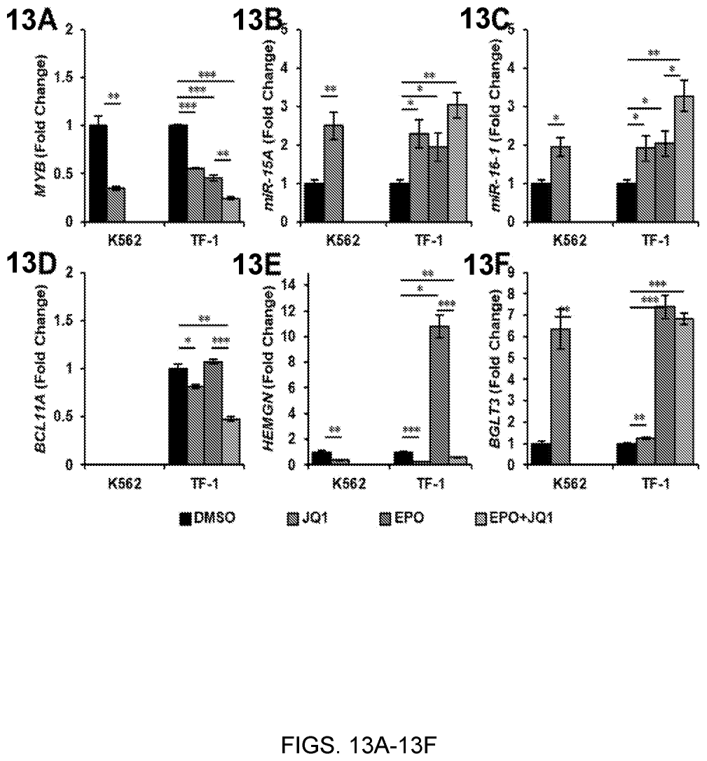

To understand the mechanisms by which BET inhibitors upregulate fetal and embryonic hemoglobin genes, the expression of known fetal hemoglobin inhibitor genes was examined via RT-qPCR. MYB is an inhibitor of fetal hemoglobin, and is downregulated by the microRNAs miR-ISA and miR-16-1 (3, 20-23). In both K562 and TF-1, MYB expression was significantly suppressed by JQ1 as well as by EPO (FIG. 13A). Consistent with this observation, the microRNAs miR-15A and miR-16-1 were significantly upregulated by both JQ1 and EPO, showing an inverse correlation with MYB expression (FIGS. 13B-13C). BCL11A is a well established inhibitor of fetal hemoglobin, and deletion of its binding site such as in patients with Corfu deltabeta thalassemia exhibit hereditary persistence of fetal hemoglobin (1, 3, 24). While BCL11A was not expressed in K562, it was significantly suppressed by JQ1 but not EPO in TF-1 cells (FIG. 13D). HEMGN encodes the protein EDAG, which promotes GATA1 acetylation by recruiting EP300 (25). It was found that HEMGN was significantly suppressed by JQ1 in both cell lines, especially in TF-1 cells under erythropoietic condition (FIG. 13E). Lastly. BGLT3 is known to promote fetal hemoglobin expression by promoting looping of HBG1/2 and the LCR (26). It was observed that JQ1 treatment increased BGLT3 expression by 6-fold in K562 cells, but not TF-1 cells, which explains in part why HBG1/2 were not induced by JQ1 in TF-1 cells.

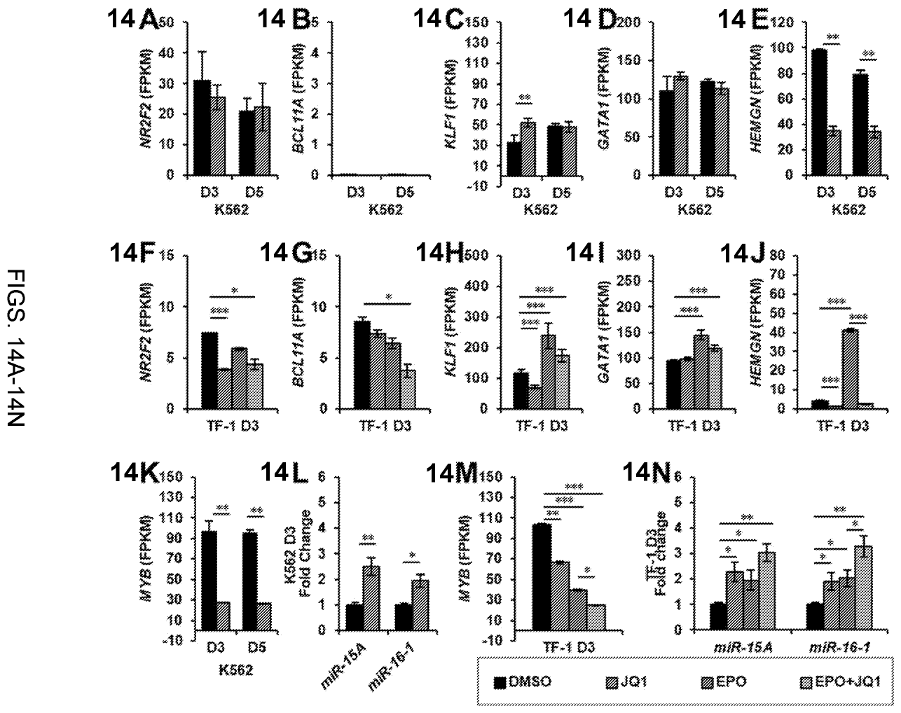

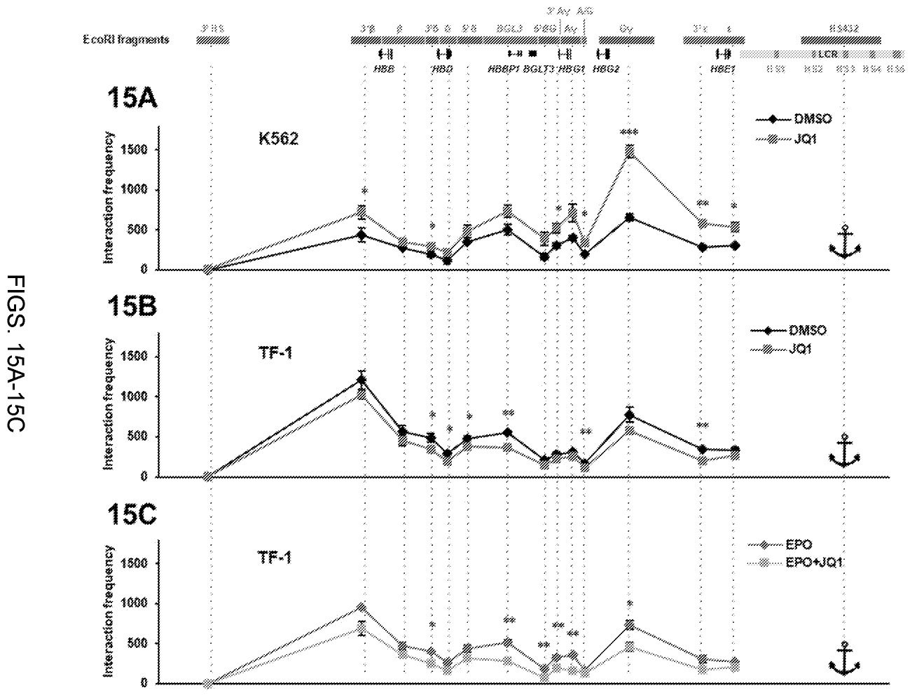

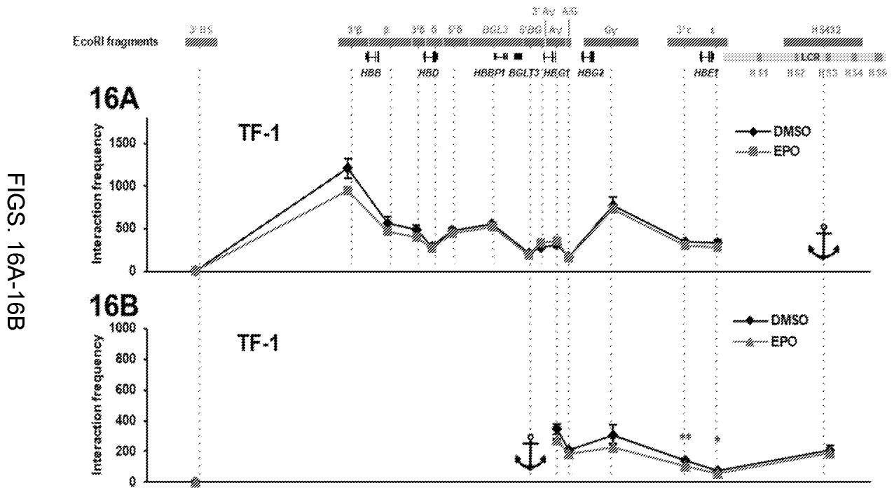

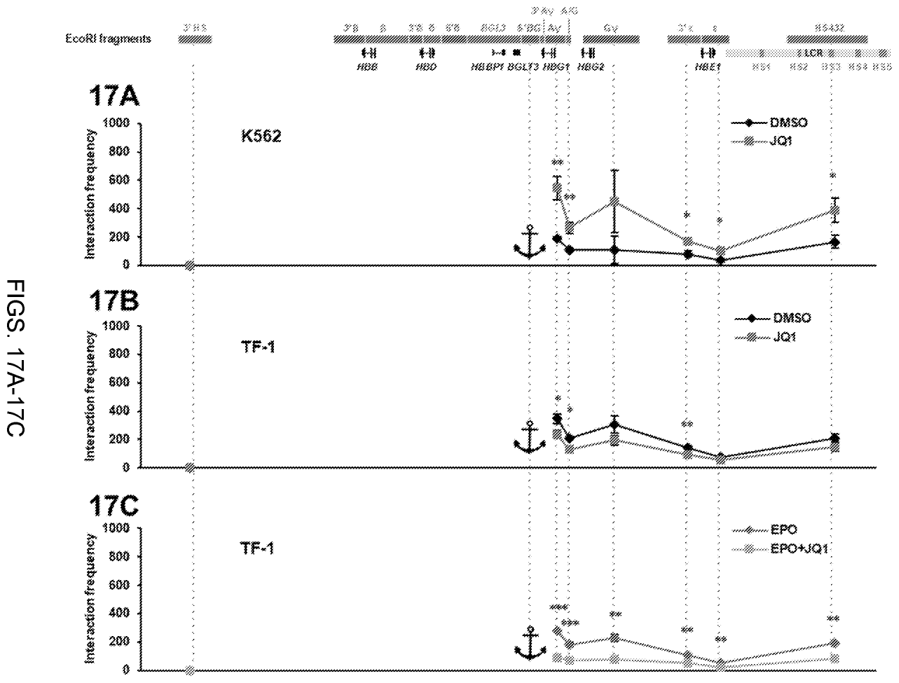

These results were validated with RNA-seq and additioned genes involved in fetal hemoglobin suppression were examined (FIGS. 14A-14N). COUP-TFII (encoded by NR2F2 gene) had been shown to inhibit fetal hemoglobin genes (27). Although JQ1 did not change NR2F2 expression in K562 cells, it did significantly suppressed NR2F2 expression in proliferative TF-1 cells (FIGS. 14A, 14F). KLF1 had been shown to activate the expression of BCL11A, and subsequently fetal hemoglobin (2, 3, 28). Since K562 cells do not express BCL11A, changes in KLF1 expression would not affect fetal hemoglobin through this pathway (FIGS. 14B-14C). In TF-1 cells, however, KLF1 was significantly suppressed by JQ1 in proliferative condition, and slightly decreased in erythropoietic condition, which may in part account for the decrease in BCL11A expression (FIGS. 14G-14H). GATA1 binds to BCL11A and NuRD complex to inhibit fetal hemoglobin expression (3). GATA1 was also shown to maintain the spatial organization of the chromatin around the β-globin locus (8). Additionally, GATA1 transcriptional activity is dependent on BET proteins, which binds to acetylated GATA1 (10, 11, 29). JQ1 treatment did not change GATA1 expression in either K562 or TF-1 cells, but significantly suppresses HEMGN in both cell lines (FIGS. 14D-14E and 14I-14J), suggesting potential change in acetylation of GATA1.

JQ1 Modulates Chromatin Spatial Organization at β-Globin Locus

Previous studies showed that expression of different β-globin genes is dependent on the contact frequency between the promoter of each gene and the LCR enhancers (5, 6). Here, chromatin conformation capture (3C) was performed to examine how the spatial structure of this locus changes in response to 72 hours of JQ1 treatment. In K562 cells, JQ1 led to over 2-fold increase in interaction frequency between the LCR and the HBG promoter region, while causing slight increases all throughout the rest of the locus (FIG. 15A). This observation correlates well with the increased expression of HBG/2 in K562 cells (FIG. 15B, FIG. 11A). In TF-1 cells, JQ1 caused modest decrease in interaction with LCR across the entire locus in both proliferative and erythropoietic conditions, except at the HBE1 promoter region (FIGS. 15B-15C). This shift in relative interaction frequency towards HBE1, combined with the linear proximity of HBE1 to the LCR enhancers, could explain the specific upregulation of embryonic but not fetal and adult hemoglobins by JQ1. In contrast, EPO treatment had only minor effect on chromatin conformation at the locus (FIGS. 16A-16B).

A recent study showed that the BGLT3 lncRNA promotes looping between fetal hemoglobin genes and the LCR (26). Therefore, the interaction between the 5′BGLT3 (5′BG) fragment and its upstream loci was examined. In K562 cells, JQ1 significantly increase the interaction frequency of 5′BG with the HBG1/2 region, as well as with the LCR (FIG. 17A), consistent with the increase in BGLT3 expression (FIG. 13F) and the increased interaction between the LCR and HBG promoter region (FIG. 15A). In TF-1 cells, JQ1 only caused slight decrease in the interaction between 5′BG and upstream fragments, which is more prominent in the erythropoietic condition (FIGS. 17B-17C). This is again consistent with the changes in BGLT3 expression (FIG. 13F) and the lack of fetal hemoglobin upregulation in TF-1 cells (FIGS. 7E, 11C).

GATA1 Protein Level and Acetylation is not Affected by JQ1

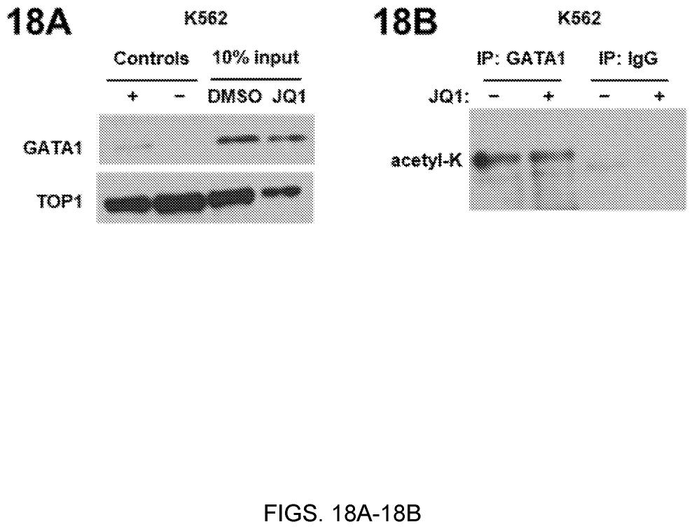

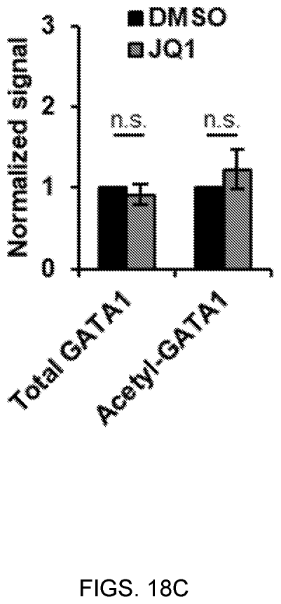

To test whether HEMGN downregulation led to decreased acetylation of GATA1, immunoprecipitation (IP) was performed on nuclear protein lysates from JQ1 treated cells along with controls. In K562 cells, JQ1 treatment did not affect the total GATA1 level (FIGS. 18A, 18C), consistent with the observation from RNA-seq (FIG. 14D). Surprisingly, GATA1 acetylation also remained unchanged by JQ1 treatment (FIGS. 18B-18C). Since GATA1 activity is dependent on the association of BET proteins (10,11,29), this result suggests that inhibition of BET recognition of acetyl-lysine, rather than the loss of GATA1 acetylation, was responsible for the changes in chromatin conformation and β-globin gene expression.

### Discussion

Hemoglobin switch in human development is a well-studied topic and have immense clinical implications. Within the β-globin locus, five hemoglobin genes are expressed under the control of LCR enhancers. Various studies have established that the LCR looping and physical proximity to each hemoglobin gene determine the expression level of these genes (1,2). In fact, forced contact between LCR and the fetal hemoglobins was shown to reactivate fetal hemoglobin expression in adult mice erythroblast (5,6). The 3D structure of the β-globin locus is maintained by many different factors. GATA1 plays a role in maintaining the overall 3D structure of the β-globin locus (8), and is the key transcription factor that promotes LCR binding to adult hemoglobin genes (7). Recent studies have shown that GATA1 binding to erythroid target genes is dependent on its acetylation and association with BET proteins (10,11,29). This raises the possibility of altering the 3D structure β-globin locus by targeting BET proteins. Indeed, previous study has demonstrated that the BET inhibitor JQ1 can promote erythropoiesis in vitro (12), suggesting that BET inhibitors are promising drugs in treating various blood disorders that involve anemia.

In this study, it was discovered that BET inhibitors specifically upregulate fetal and embryonic hemoglobin genes, but had no effect on or even suppressed adult hemoglobin in multiple erythroid cell lines. The findings here has great implications in treating inherited blood disorders involving mutations of the adult hemoglobin genes, notably sickle cell anemia (SCA) and β-thalassemia. SCA is the most common inherited disorder in the United States, and is especially prevalent in many parts of Africa where malaria is common. The most common treatment for SCA is hydroxyurea, which elevates fetal hemoglobin production to alleviate the symptoms. In late 2019, two additional drugs were FDA-approved to treat SCA, voxelotor and crizanlizumab. Voxelotor enhances the affinity of hemoglobin to oxygen, reducing the risk of sickle hemoglobin polymerization in low-oxygen parts of the body (30). Crizanlizumab is an monoclonal antibody against P-selectin, which plays a major part in initiating vaso-occlusive crisis, a common clinical manifestation of SCA (30). Both of these drugs tackle the symptoms caused by sickle cell hemoglobin without reducing the mutant hemoglobin. Although the recent advances in gene therapies made it possible to cure SCA, the high costs for such procedures make them unaffordable for may low-income families. The current findings suggest that the various BET inhibitors, many of which already in clinical trial for blood related disorders, may be suitable addition to the options in treating SCA and other disorders caused by mutations of the HBB gene.

In TF-1 cells, it was found that JQ1 only upregulates the embryonic hemoglobin gene HBE1. One important question with clinical implication is whether the HBE1 encoded ε-globin can be a reasonable substitute for normal β-globin. Biochemical analysis on the semi-embryonic hemoglobin Hb-Gower 2 (α₂ε₂) showed that this its P₅₀ for oxygen, affinity to 2,3-BPG, Bohr Coefficient, and Hill Coefficient are comparable to those of HbA (α₂β₂)(31). Hb-Gower 2 also has a comparable tetramer-dimer disassociation constant to that of HbA (32). A study in transgenic α/β-thalassemia mice found that human embryonic hemoglobins HBZ1 (ζ-globin) and HBE1 (ε-globin) rescue the lethal phenotype of α/β-thalassemia (33). Similarly, a study in sickle cell mice found that the presence of human Hb-Gower 2 greatly alleviated sickle cell phenotypes, and found that Hb-Gower 2 inhibits HbS polymerization (34). Taken together, these studies indicate that reactivation of either fetal or embryonic hemoglobin is beneficial in treating SCA and β-thalassemia.

In conclusion, this study demonstrates that the pharmacological inhibition of BET proteins could significantly alter the composition of hemoglobins, skewing from adult-towards fetal-/embryonic-hemoglobin. Multiple BET inhibitors of different structures were shown to produce similar effects in erythroid cell lines, which suggest that the effects may apply to most, if not all, members in the BET inhibitor family. There are many different BET inhibitors currently under clinical trials for various diseases (35, 36), and the current results showed that patients with SCA or β-thalassemia may benefit from BET inhibition.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### REFERENCES

1. Sankaran V G, Orkin S H. The switch from fetal to adult hemoglobin. Cold Spring Harbor perspectives in medicine. 2013; 3(1).
2. Wilber A, Nienhuis A W, Persons D A. Transcriptional regulation of fetal to adult hemoglobin switching: new therapeutic opportunities. Blood. 2011; 117(15):3943-3953.
3. Paikari A, Sheehan V A. Fetal haemoglobin induction in sickle cell disease. British journal of haematology. 2018; 180(2):189-200.
4. Sankaran V G. Xu J, Orkin S H. Advances in the understanding of haemoglobin switching. British journal of haematology. 2010; 149(2):181-194.
5. Deng W, Lee J, Wang H, et al. Controlling long-range genomic interactions at a native locus by targeted tethering of a looping factor. Cell. 2012; 149(6):1233-1244.
6. Deng W, Rupon J W, Krivega I, et al. Reactivation of developmentally silenced globin genes by forced chromatin looping. Cell. 2014; 158(4):849-860.
7. Vakoc C R, Letting D L, Gheldof N, et al. Proximity among distant regulatory elements at the beta-globin locus requires GATA-1 and FOG-1. Molecular cell. 2005; 17(3):453-462.
8. Kang Y, Kim Y W, Kang J, Yun W J, Kim A. Erythroid specific activator GATA-1-dependent interactions between CTCF sites around the β-globin locus. Biochimica et biophysica acta Gene regulatory mechanisms. 2017; 1860(4):416-426.
9. Kang Y, Kim Y W, Yun J, Shin J, Kim A. KLF1 stabilizes GATA-1 and TAL1 occupancy in the human β-globin locus. Biochimica et biophysica acta. 2015; 1849(3):282-289.
10. Lamonica J M, Deng W, Kadauke S, et al. Bromodomain protein Brd3 associates with acetylated GATA1 to promote its chromatin occupancy at erythroid target genes. Proceedings of the National Academy of Sciences of the United States of America. 2011; 108(22):68.
11. Stonestrom A J, Hsu S C, Jahn K S, et al. Functions of BET proteins in erythroid gene expression. Blood. 2015; 125(18):2825-2834.
12. Goupille O, Penglong T, Leftvre C, et al. BET bromodomain inhibition rescues erythropoietin differentiation of human erythroleukemia cell line UT7. Biochemical and biophysical research communications. 2012; 429(1-2):1-5.
13. Liu X, Zhang Y, Chen Y, et al. In Situ Capture of Chromatin Interactions by Biotinylated dCas9. Cell. 2017; 170(5):1028.
14. Rathert P, Roth M, Neumann T, et al. Transcriptional plasticity promotes primary and acquired resistance to BET inhibition. Nature. 2015; 525(7570):543-547.
15. Sdelci S, Rendeiro A F F, Rathert P, et al. MTHFD1 interaction with BRD4 links folate metabolism to transcriptional regulation. Nature genetics. 2019; 51(6):990-998.
16. Garcia-Carpizo V, Ruiz-Llorente S, Sarmentero J, Graña-Castro O, Pisano D G, Barrero M J. CREBBP/EP300 bromodomains are critical to sustain the GATA1/MYC regulatory axis in proliferation. Epigenetics & chromatin. 2018; 11(1):30.
17. Filippakopoulos P, Qi J, Picaud S, et al. Selective inhibition of BET bromodomains. Nature. 2010; 468(7327):1067-1073.
18. Albrecht B K, Gehling V S, Hewitt M C, et al. Identification of a Benzoisoxazoloazepine Inhibitor (CPI-0610) of the Bromodomain and Extra-Terminal (BET) Family as a Candidate for Human Clinical Trials. Journal of medicinal chemistry. 2016; 59(4):1330-1339.
19. Ozer H G, El-Gamal D, Powell B, et al. BRD4 Profiling Identifies Critical Chronic Lymphocytic Leukemia Oncogenic Circuits and Reveals Sensitivity to PLX51107, a Novel Structurally Distinct BET Inhibitor. Cancer discovery. 2018; 8(4):458-477.
20. Stadhouders R, Aktuna S. Thongjuea S, et al. HBS1L-MYB intergenic variants modulate fetal hemoglobin via long-range MYB enhancers. The Journal of clinical investigation. 2014; 124(4):1699-1710.
21. Wang X, Angelis N, Thein S L. MYB—A regulatory factor in hematopoiesis. Gene. 2018; 665:6-17.
22. Sankaran V G, Menne T F, Šćepanović D, et al. MicroRNA-15a and -16-1 act via MYB to elevate fetal hemoglobin expression in human trisomy 13. Proceedings of the National Academy of Sciences of the United States of America. 2011; 108(4):1519-1524.
23. Zhao F H, Kalota A, Jin S, Gewirtz A M. The c-myb prot-oncogene and microRNA-15a comprise an active autoregulatory feedback loop in human hematopoietic cells. Blood. 2009; 113(3):505-516.
24. Chakalova L, Osborne C S, Dai Y, et al. The Corfu deltabeta thalassemia deletion disrupts gamma-globin gene silencing and reveals post-transcriptional regulation of HbF expression. Blood. 2005; 105(5):2154-2160.
25. Zheng W, Dong X, Yin R, et al. EDAG positively regulates erythroid differentiation and modifies GATA1 acetylation through recruiting p300. Stem cells (Dayton, Ohio). 2014; 32(8):2278-2289.
26. Ivaldi M S, Diaz L F, Chakalova L, Lee J, Krivega I, Dean A. Fetal γ-globin genes are regulated by the BGLT3 long noncoding RNA locus. Blood. 2018; 132(18):1963-1973.
27. Aerbajinai W, Zhu J, Kumkhaek C, Chin K, Rodgers G P. SCF induces gamma-globin gene expression by regulating downstream transcription factor COUP-TFII. Blood. 2009; 114(1):187-194.
28. Fanis P, Kousiappa I, Phylactides M, et al. A novel mutation in the erythroid transcription factor KLF1 is likely responsible for ameliorating β-thalassemia major. Human mutation. 2019; 40(10):1768-1780.
29. Gamsjaeger R, Webb S R, Lamonica J M, Billin A, Blobel G A, Mackay J P. Structural basis and specificity of acetylated transcription factor GATA1 recognition by BET family bromodomain protein Brd3. Molecular and cellular biology. 2011; 31(13):2632-2640.
30. Ali M A, Ahmad A, Chaudry H, et al, Efficacy and safety of recently approved drugs for sickle cell disease: a review of clinical trials. Experimental hematology. 2020.
31. He Z, Russell J E. Expression, purification, and characterization of human hemoglobins Gower-1 (zeta(2)epsilon(2)), Gower-2 (alpha(2)epsilon(2)), and Portland-2 (zeta(2)beta(2)) assembled in complex transgenic-knockout mice. Blood. 2001; 97(4):1099-1105.
32. Manning L R, Russell J E, Padovan J C, et al. Human embryonic, fetal, and adult hemoglobins have different subunit interface strengths. Correlation with lifespan in the red cell. Protein science: a publication of the Protein Society. 2007; 16(8):1641-1658.
33. Russell J E, Liebhaber S A. Reversal of lethal alpha- and beta-thalassemias in mice by expression of human embryonic globins. Blood. 1998; 92(9):3057-3063.
34. He Z, Russell J E. A human embryonic hemoglobin inhibits Hb S polymerization in vitro and restores a normal phenotype to mouse models of sickle cell disease. Proceedings of the National Academy of Sciences of the United States of America. 2002; 99(16):10635-10640.
35. Alqahtani A, Choucair K, Ashraf M, et al. Bromodomain and extra-terminal motif inhibitors: a review of preclinical and clinical advances in cancer therapy. Future science OA. 2019; 5(3).
36. Doroshow D B, Eder J P, LoRusso P M. BET inhibitors: a novel epigenetic approach. Annals of oncology: official journal of the European Society for Medical Oncology. 2017; 28(8):1776-1787.
37. Ruifrok A C, Johnston D A. Quantification of histochemical staining by color deconvolution. Analytical and quantitative cytology and histology. 2001; 23(4):291-299.
38. Kim D. Paggi J M, Park C, Bennett C, Salzberg S L. Graph-based genome alignment and genotyping with HISAT2 and HISAT-genotype. Nature biotechnology. 2019; 37(8):907-915.
39. Frankish A, Diekhans M, Ferreira A-MM, et al. GENCODE reference annotation for the human and mouse genomes. Nucleic acids research. 2019; 47(D1).
40. Trapnell C, Roberts A, Goff L, et al. Differential gene and transcript expression analysis of RNA-seq experiments with TopHat and Cufflinks. Nature protocols. 2012; 7(3):562-578.
41. Reimand J, Isserlin R, Voisin V, et al. Pathway enrichment analysis and visualization of omics data using g:Profiler, GSEA, Cytoscape and EnrichmentMap. Nature protocols. 2019; 14(2):482-517.
42. Hagège H, Klous P, Braem C, et al. Quantitative analysis of chromosome conformation capture assays (3C-qPCR). Nature protocols. 2007; 2(7):1722-1733.
43. Kiefer C M, Lee J, Hou C, et al. Distinct Ldb1/NLI complexes orchestrate γ-globin repression and reactivation through ETO2 in human adult erythroid cells. Blood. 2011; 118(23):6200-6208.
44. Morgan S I., Mariano N C, Bermudez A, et al. Manipulation of nuclear architecture through CRISPR-mediated chromosomal looping. Nature communications. 2017; 8:15993.
45. Camenisch G, Wenger R H, Gassmann M. DNA-binding activity of hypoxia-inducible factors (HIFs). Methods in molecular biology (Clifton, N.J.). 2002; 196:117-129.

TABLE 1
Sequences.

| | | SEQ ID |
|---|---|---|
| Name | Sequence | NO: |
| HBG1 partial sequence 1 | TGCCACAAAGC | 1 |
| HBG2 partial sequence 1 | TGCCATAAAGC | 2 |
| HBG1 partial sequence 2 | GACTGCAGTGG | 3 |
| HBG2 partial sequence 2 | GACTGGAGTGG | 4 |
| HBG1 partial sequence 3 | ATGATTCAGAG | 5 |
| HBG2 partial sequence 3 | ATGATGCAGAG | 6 |
| HBB forward | GGCTCACCTGGACAACCTCA | 7 |
| HBB reverse | AAAGTGATGGGCCAGCACACAG | 8 |
| HBG1/2 forward (qPCR) | AAAGCACCTGGATGATCTCA | 9 |
| HBG1/2 reverse (qPCR) | AAAACGGTCACCAGCACATTT | 10 |
| HBG1/2 forward | CACATGGCAAGAAGGTGCT | 11 |
| (sequencing) | | |
| HBG1/2 reverse | GCAGAATAAAGCCTATCCTTGAAA | 12 |
| (sequencing) | | |
| HBE1 forward | CAAGCCCGCCTTTGCTAAGCT | 13 |
| HBE1 reverse | CTCCTTGCCAAAGTGAGTAGCCAG | 14 |
| 18S forward | GAGGGAGCCTGAGAAACGG | 15 |
| 18S reverse | GTCGGGAGTGGGTAATTTGC | 16 |
| MYB F | TCCCAAGTCTGGAAAGCGTC | 17 |
| MYB R | GCACATCTGTTCGATTCGGG | 18 |
| BCL11A F | TGCCCCAAACAGGAACACAT | 19 |
| BCL11A R | ATTCTGCACTCATCCCAGGC | 20 |
| HEMGN F | AAGTCATTGGAACCTGGAGTTTG | 21 |
| HEMGN R | GTTCTCTGCTGCTTGCGTTT | 22 |
| BGLT3 F | TCACTGGTACGCAGGGTTTT | 23 |
| BGLT3 R | TATTGAGTTGTGGGGACTGGC | 24 |
| 3′HS1 | ATTCCCGTTTTTATGAAATCAACTTT | 25 |
| B/HS | TCTTAGAAAGCCTTTACAATTTCCTTTATC | 26 |
| 3′Beta | AGCTTAGTGATACTTGTGGGCCA | 27 |
| Beta | GCTCGGCACATGTCCCATCCAG | 28 |
| DB1 | GTCAGTGAGTCTAGGCAAGATGTTGGCC | 29 |
| Delta | AAAAAATGTGGAATTAGACCCAGGAATG | 30 |
| 5′Delta | GGGTGTGTATTTGTCTGCCA | 31 |
| BGL3 | TTGCCATACCTCATATCCTTAG | 32 |
| 5′BGL3 | CTTAGGCATCCACAAGGG | 33 |
| G/BGL3 | AGCAAGGATGGTTCTTAAGGAAGGG | 34 |
| Ag | ATCCATGATCTCTAACCTTGC | 35 |
| G/A | AATTTGAAGATACAGCTTGCCTCCGATAAG | 36 |
| Gg | GGGTTCATCTTTATTGTCTCCT | 37 |
| E/G | CCACCCCGATAAAGATTTTTCTCCATCA | 38 |
| Epsilon | ATTAACCAATGGTATCTTTCTGAGCA | 39 |
| HS432 | CCAAATGGGTGACTGTAGGGTTGAGA | 40 |
| B13 F | CGTGAGAGCATACTTCCTGGTTC | 41 |
| B13 R | ACACCAGAGAGGTCTTGCCCT | 42 |

TABLE 2
3C-qPCR primer pairs.

| | | |
|---|---|---|
| Name | Forward | Reverse |

Digestion efficiency primer pairs

| | | |
|---|---|---|
| No digestion control | B13 F | B13 R |
| 3′HS site | 3′HS1 | B/HS |
| HBB site | Beta | DB1 |
| HBD site | Delta | 5′Delta |
| BGLT3 site | 5′BGL3 | G/BGL3 |
| HBG1/2 site | Ag | G/A |

Purity test primer pairs

| | | |
|---|---|---|
| No digestion control | B13 F | B13 R |

Concentration primer pairs

| | | |
|---|---|---|
| No digestion control | B13 F | B13 R |

3C primer pairs

| | | |
|---|---|---|
| No digestion control | B13 F | B13R |
| 3′HS | HS432 | 3′HS1 |
| 3′Beta | HS432 | 3′Beta |
| Beta | HS432 | Beta |
| Beta/Delta | HS432 | DB1 |
| Delta | HS432 | Delta |
| 5′ Delta | HS432 | 5′Delta |
| BGLT3 | HS432 | BGE3 |
| 5′BGLT3 | HS432 | 5′BGL3 |
| 3′Gamma1 | HS432 | G/BGL3 |
| Gamma1 | HS432 | Ag |
| 3′Gamma2 | HS432 | G/A |
| Gamma1/2 | HS432 | Gg |
| 3′Epsilon | HS432 | E/G |
| Epsilon | HS432 | Epsilon |
| BGLT3-3′HS | 5′BGL3 | 3′HS1 |
| BGLT3-Gamma1 | 5′BGL3 | Ag |
| BGLT3-3′Gamma2 | 5′BGL3 | G/A |
| BGLT3-Gamma1/2 | 5′BGL3 | Gg |
| BGLT3-3′Epsilon | 5′BGL3 | E/G |
| BGLT3-Epsilon | 5′BGL3 | Epsilon |

1. A method of treating an inherited blood disorder in a subject in need thereof, comprising administering to the subject a therapeutically effective amount of a bromodomain and extra-terminal motif (BET) protein inhibitor, wherein the therapeutically effective amount of the BET protein inhibitor induces expression of fetal and/or embryonic hemoglobin in the subject.

2. The method of claim 1, wherein the inherited blood disorder is thalassemia.

3. The method of claim 2, wherein the thalassemia is alpha thalassemia or beta thalassemia.

4. (canceled)

5. The method of claim 1, wherein the inherited blood disorder is sickle cell anemia.

6. The method of claim 5, wherein the sickle cell anemia is type HbSS, HbSC, HbS beta thalassemia, HbSD, HbSE, or HbSO.

7-9. (canceled)

10. The method of claim 1, wherein the BET protein inhibitor is JQ1, CPI-0610, and/or PLX51107.

11-13. (canceled)

14. The method of claim 1, wherein the BET protein inhibitor inhibits bromodomain-containing protein 2 (BRD2), bromodomain-containing protein 3 (BRD3), bromodomain-containing protein 4 (BRD4), and/or bromodomain testis-specific protein (BRDT).

15-16. (canceled)

17. The method of claim 1, wherein the BET protein inhibitor is administered to the subject in conjunction with a second therapy.

18. The method of claim 17, wherein the second therapy comprises a histone deacetylase (HDAC) inhibitor, an antineoplastic agent, an HbS polymerization inhibitor, and/or a gene therapy.

19. (canceled)

20. The method of claim 18, wherein the antineoplastic agent comprises hydroxyurea.

21. (canceled)

22. The method of claim 18, wherein the HbS polymerization inhibitor is voxelotor.

23. (canceled)

24. The method of claim 18, wherein the gene therapy utilizes a lentiviral vector and/or gene editing.

25. (canceled)

26. A method of inducing expression of fetal and/or embryonic hemoglobin in a subject, comprising administering to the subject a bromodomain and extra-terminal motif (BET) protein inhibitor to the subject.

27. The method of claim 26, wherein the fetal hemoglobin is hemoglobin F (HbF).

28. The method of claim 26, wherein the embryonic hemoglobin is embryonic hemoglobin (HbE).

29. The method of any of claims 26-28, wherein the BET protein inhibitor is JQ1, CPI-0610, and/or PLX51107.

30-32. (canceled)

33. The method of claim 26, wherein the BET protein inhibitor inhibits bromodomain-containing protein 2 (BRD2), bromodomain-containing protein 3 (BRD3), bromodomain-containing protein 4 (BRD4), and/or bromodomain testis-specific protein (BRDT).

34-36. (canceled)

37. The method of claim 1, wherein the expressed fetal and/or embryonic hemoglobin alleviates one or more symptoms of an inherited blood disorder in the subject comprising reduced clotting, excessive bleeding, fatigue, dizziness, malaise, anemia, joint pain, chest pain, delayed development, jaundice, or dactylitis.

38. (canceled)

39. A method of inducing erythropoiesis in a subject, comprising administering to the subject a therapeutically effective amount of a bromodomain and extra-terminal motif (BET) protein inhibitor to the subject.

40. The method of claim 39, wherein the BET protein inhibitor is one or more of JQ1, I-BET 151, I-BET 762, OTX-015, TEN-010, CPI-203, CPI-0610, olinone, RVX-208, LY294002, AZD5153, MT-1, MS645, RVX-297, PLX51107, BMS-986158, FT-1101, INCB054329, INCB057643, and ZEN-3694.
